SEQUENCE LISTING (16 sequences)

SEQ ID NO: 1 - DNA, 20 bp, Artificial Sequence
  Oligonucleotide primer
cgccgctagaggtgaaattc

SEQ ID NO: 2 - DNA, 18 bp, Artificial Sequence
  Oligonucleotide primer
ttggcaaatgctttcgct

SEQ ID NO: 3 - DNA, 21 bp, Artificial Sequence
  Oligonucleotide primer
cctcatcccgtgttctccttt

SEQ ID NO: 4 - DNA, 20 bp, Artificial Sequence
  Oligonucleotide primer
gtaccacccagcggacaagt

SEQ ID NO: 5 - DNA, 21 bp, Artificial Sequence
  Oligonucleotide primer
atttcttcggtcactacacaa

SEQ ID NO: 6 - DNA, 18 bp, Artificial Sequence
  Oligonucleotide primer
aacgcccaacaggaacac

SEQ ID NO: 7 - DNA, 18 bp, Artificial Sequence
  Oligonucleotide primer
ctcaacacgcctcatcct

SEQ ID NO: 8 - DNA, 20 bp, Artificial Sequence
  Oligonucleotide primer
gtgctcgctcatgtaattgc

SEQ ID NO: 9 - DNA, 24 bp, Artificial Sequence
  Oligonucleotide primer
tccttgtcagaatccattacctgt

SEQ ID NO: 10 - DNA, 21 bp, Artificial Sequence
  Oligonucleotide primer
gaatgcgctgggaagaatttg

SEQ ID NO: 11 - DNA, 21 bp, Artificial Sequence
  Oligonucleotide primer
agagtgcatcacacaacgaga

SEQ ID NO: 12 - DNA, 23 bp, Artificial Sequence
  Oligonucleotide primer
ctgagcagtttggagatgaggtc

SEQ ID NO: 13 - DNA, 20 bp, Artificial Sequence
  Oligonucleotide primer
agtgtgacccagactgcctc

SEQ ID NO: 14 - DNA, 20 bp, Artificial Sequence
  Oligonucleotide primer
caagccaggtccacctcctc

SEQ ID NO: 15 - DNA, 21 bp, Artificial Sequence
  Oligonucleotide primer
ttggtctggaggtcttggaga

SEQ ID NO: 16 - DNA, 23 bp, Artificial Sequence
  Oligonucleotide primer
gaatcgctctttcttcggaactg

(19) United States
(12) Patent Application Publication
(10) Pub. No.: US 2009/0209606 A1
(43) Pub. Date: Aug. 20, 2009

(54) Cancer Treatments

(76) Inventors: Heather Helene Bendall, Del Mar, CA (US); Gary T. Elliott, San Diego, CA (US); Lorenzo M. Leoni, Lodrino (CH); Christina Carol Niemeyer, Poway, CA (US); Pratik S. Multani, San Diego, CA (US)

Correspondence Address: WOODCOCK WASHBURN LLP, CIRA CENTRE, 12TH FLOOR, 2929 ARCH STRET, PHILADELPHIA, PA, 19104-2891, US

(21) Appl. No.: 12/372,910

(22) Filed: Feb. 18, 2009

Related U.S. Application Data
(62) Division of application No. 11/267,010, filed on Nov. 4, 2005, now abandoned.
(60) Provisional application No. 60/660,226, filed on Mar. 10, 2005.
(60) Provisional application No. 60/625,193, filed on Nov. 5, 2004.

(51) Int. Cl. A61K 31/4184; A61P 35/00

(52) U.S. Cl. 514/394

(57) ABSTRACT

Methods and compositions for treating cancers characterized by death-resistant cancer cells are described. In general, such methods involve administration of a therapeutically effective amount of a compound that induces mitotic catastrophe in the some, and preferably most or all, of the cancerous cells. Methods for assessing the efficacy of such treatments are also provided.

### CROSS REFERENCE TO RELATED APPLICATIONS

This application is a divisional of U.S. application Ser. No. 11/267,010, filed Nov. 4, 2005, which claims the benefit of U.S. Provisional Application No. 60/625,193, filed Nov. 5, 2004; and U.S. Provisional Application No. 60/660,266, filed Mar. 10, 2005. Each of these applications is incorporated herein by reference in its entirety.

### TECHNICAL FIELD

This invention relates generally to cancer treatment, particularly cancers resistant to drug-induced apoptosis.

### BACKGROUND

The following description includes information that may be useful in understanding the present invention. It is not an admission that any such information is prior art, or relevant, to the presently claimed inventions, or that any publication specifically or implicitly referenced is prior art.

Cancer is now the second leading cause of death in the United States and over 8,000,000 persons in the United States have been diagnosed with cancer. In 1995, cancer accounted for 23.3% of all deaths in the United States. See U.S. Dept. of Health and Human Services, National Center for Health Statistics, Health United States 1996-97 and Injury Chartbook 117 (1997).

Cancer is not fully understood on the molecular level. It is known that exposure of a cell to a carcinogen such as certain viruses, certain chemicals, or radiation, leads to DNA alteration that inactivates a “suppressive” gene or activates an “oncogene”. Suppressive genes are growth regulatory genes, which upon mutation, can no longer control cell growth. Oncogenes are initially normal genes (called proto-oncogenes) that by mutation or altered context of expression become transforming genes. The products of transforming genes cause inappropriate cell growth. More than twenty different normal cellular genes can become oncogenes by genetic alteration. Transformed cells differ from normal cells in many ways, including cell morphology, cell-to-cell interactions, membrane content, cytoskeletal structure, protein secretion, gene expression and mortality (transformed cells can grow indefinitely).

A neoplasm, or tumor, is an abnormal, unregulated, and disorganized proliferation of cell growth, and is generally referred to as cancer. A neoplasm is malignant, or cancerous, if it has properties of destructive growth, invasiveness, and metastasis. Invasiveness refers to the local spread of a neoplasm by infiltration or destruction of surrounding tissue, typically breaking through the basal laminas that define the boundaries of the tissues, thereby often entering the body's circulatory system. Metastasis typically refers to the dissemination of tumor cells by lymphatics or blood vessels. Metastasis also refers to the migration of tumor cells by direct extension through serous cavities, or subarachnoid or other spaces. Through the process of metastasis, tumor cell migration to other areas of the body establishes neoplasms in areas away from the site of initial appearance.

Cancer is now primarily treated with one or a combination of three types of therapies: surgery; radiation; and chemotherapy. Surgery involves the bulk removal of diseased tissue. While surgery is sometimes effective in removing tumors located at certain sites, for example, in the breast, colon, and skin, it cannot be used in the treatment of tumors located in other areas, such as the backbone, nor in the treatment of disseminated neoplastic conditions such as leukemia. Radiation therapy involves the exposure of living tissue to ionizing radiation causing death or damage to the exposed cells. Side effects from radiation therapy may be acute and temporary, while others may be irreversible. Chemotherapy involves the disruption of cell replication or cell metabolism. It is used most often in the treatment of breast, lung, and testicular cancer.

The adverse effects of systemic chemotherapy used in the treatment of neoplastic disease are most feared by patients undergoing treatment for cancer. Of these adverse effects, nausea and vomiting are the most common. Other adverse side effects include cytopenia, infection, cachexia, mucositis in patients receiving high doses of chemotherapy with bone marrow rescue or radiation therapy; alopecia (hair loss); cutaneous complications such as pruritis, urticaria, and angioedema; neurological complications; pulmonary and cardiac complications; and reproductive and endocrine complications. Chemotherapy-induced side effects significantly impact the quality of life of the patient and may dramatically influence patient compliance with treatment. As such, improved methods of treatment are needed.

### SUMMARY

One object of this invention is to provide patentable methods of treating cancers characterized by death-resistant cancer cells by administration of a compound (e.g., bendamustine) that induces mitotic catastrophe in the cancer cells, alone or in conjunction with other compounds and/or treatments. In preferred embodiments, these methods involve determining whether a patient has a cancer characterized by death-resistant cancer cells, and, if so, then administering to the patient a therapeutically effective amount of bendamustine. Still another object of the invention concerns methods of assessing the efficacy of cancer treatments based on the detection of a cancer cell death marker in a biological sample taken from a patient at one or more periods during or after the administration of a cancer therapy.

Thus, one aspect of the invention relates to patentable methods of treating cancer patients whose cancers are characterized by death-resistant cancer cells, i.e., cancer cells that resist apoptosis or other programmed cell death pathways, as well as cells that exhibit multi-drug resistance (MDR), as may be induced, for example, by administration of one or more alkylating agents, alone or in conjunction with an anti-CD20 agent, e.g., rituximab. These methods comprise administering to a patient a therapeutically effective amount of a compound that induces mitotic catastrophe in the death-resistant cancer cells. Such cells include those that are resistant to drug-induced apoptosis. Examples of such cells include those that have a p53 deficiency, typically as a result of a mutation of, including deletions in or of, a gene encoding p53. Representative examples of such cancers include non-Hodgkin's lymphoma (“NHL”) and chronic lymphocytic leukemia (“CLL”). A particularly preferred compound for inducing mitotic catastrophe is the alkylating agent bendamustine. Thus, a related aspect concerns methods of treatment that involve characterization of the cells of a particular cancer as death-resistant cancer cells, followed by treatment with a compound (e.g., bendamustine) that induces mitotic catastrophe in such cells, alone or in conjunction with other chemotherapeutic agents, adjuvants, surgery, and/or radiation. In addition, the efficacy of such treatment regimens can be monitored to assess whether the particular monotherapy or combination therapy treatment is achieving the desired effect.

Another aspect of the invention concerns certain related patentable methods for treating a cancer, particularly cancers characterized by death-resistant cancer cells. These methods comprise the administration to a patient of a therapeutically effective amount of a compound at a time when at least a portion of the cells comprising the cancer are in the S phase of the cell cycle. In some embodiments, at least a portion of the patient's cancerous cells are driven into the S phase as a result of administering to the patient a compound that drives cells into the S phase. Bendamustine is a particularly preferred compound for driving cancer cells into the S phase. Because bendamustine is useful in driving cancer cells into the S phase, additional preferred embodiments involve the subsequent administration of one or more other chemotherapeutic agent species that are more active (i.e., exert a greater therapeutic effect, for example, cytotoxicity, when cells are in the S-phase of the cell cycle. In such methods, the subsequent administration of one or more other chemotherapeutic agents preferably occurs at least about 10 minutes, and preferably at least about 30 to about 60 minutes or more after bendamustine administration, although it is preferred that the administration of such other agent(s) occurs within about 72 hours, preferably about 48 hours or less, after bendamustine is administered. In some of these preferred embodiments, the other chemotherapeutic agent(s) is(are) given within about 30 minutes to about 36 hours after the administration of bendamustine, preferably within about 30 minutes to 24 hours after administration of bendamustine, and in some cases, within about 30 minutes to six to about twelve hours after administration of bendamustine. Related methods involve reducing toxicity associated with a cancer therapy. Such methods comprise administering a plurality of doses of therapeutically effective amounts bendamustine to a cancer patient. The first dose may well result in an undesired toxicity. In such event, the administration of the second (or other subsequent doses) may be delayed until after the undesired toxicity begins to subside. In some cases, the doses of bendamustine administered at different times may also vary.

Yet another aspect of the invention thus relates to patentable methods for assessing the efficacy of a cancer treatment based on the administration of an alkylating agent (e.g., bendamustine), either during the course of or after completion of the treatment, be it a monotherapy or a combination therapy. When the assessment is performed after administration of a therapeutic regimen that involves administration of an alkylating agent (e.g., bendamustine), preferably a sufficient period is allowed to elapse so that the alkylating agent can exert its intended, or desired, therapeutic effect. In such methods, a marker of cancer cell death (i.e., a molecule (e.g., a protein, carbohydrate, lipid, nucleic acid, or other molecule) produced by or released from a dying or dead cancer cell, as well as a phenotype such as a lack of cell viability, inability to proliferate, senescence, etc.) that correlates with treatment efficacy is detected in a biological sample obtained from the patient to determine if the treatment with was efficacious. Preferred markers of cell death include adenylate kinase activity levels, the level of PARP cleavage products, and reduced cell viability. Depending on the marker, such detection may be qualitative, semi-quantitative, or quantitative. The presence, or level, of the marker detected indicates whether the treatment is, or has been, efficacious.

In still another aspect of the invention, the invention concerns treatments for cancer based on administering bendamustine to patients who have a cancer resistant, or refractory, to one or more alkylating agents and an anti-CD20 agent (for example, rituximab). Preferably, these methods are deployed against cancers characterized by death-resistant cancer cells. A related aspect of the invention concerns methods of doing business in the treatment of such cancers, which involve promoting bendamustine use to treat a refractory cancer or a cancer characterized by death-resistant cancer cells, particularly a cancer refractory to treatment with a combination of one or more alkylating agents and an anti-CD20 agent, e.g., rituximab. Still another aspect concerns whether a patient's cancer is amenable to bendamustine treatment. As will be appreciated, any suitable assessment of bendamustine susceptibility can be employed. In some preferred embodiments of these methods, some or all of a cell sample from cancerous tissue taken from a patient is exposed to bendamustine under growth conditions which, in the absence of a compound that is toxic to cancer cells, allows the cancer cells to proliferate. The assessment of susceptibility is then made based on the results of the assay. For example, reduced proliferation, as compared to controls, would indicate that the cells, and hence the patient's cancer, are susceptible to a bendamustine-based therapy. In contrast, no effect on (or enhanced proliferation) would indicate a lack of susceptibility.

Yet another aspect of the invention relates to the use of bendamustine in the manufacture of a medicament for treatment of a cancer characterized by death-resistant cancer cells or for treatment of a refractory cancer, particularly a cancer refractory to treatment with a combination of one or more alkylating agents and an anti-CD20 agent e.g., rituximab. Preferably, such medicaments include a therapeutically effective amount of bendamustine.

### BRIEF DESCRIPTION OF THE DRAWINGS

This patent application contains at least one figure executed in color. Copies of this patent application with color drawing(s) will be provided upon request and payment of the necessary fee.

FIG. 1 has two panels, A and B, each which show gene expression profiles. The panels show changes in gene expression measured in the Non-Hodgkin's Lymphoma cell line, SU-DHL-1, using an Affymetrix gene chip (U133A) containing more than 12,000 known genes. Bendamustine was tested at IC₅₀ (25 μM; lane 1) and IC₉₀ (35 μM; lane 2). Chlorambucil (5 μM; lane 3) and phosphoramide mustard, a cyclophosphamide metabolite (50 μM; lane 4), were tested at IC₉₀. Isolation of mRNA was performed 8 h after exposure. A. The clustergram shown represents the top 100 most modulated genes as compared to a control (diluent, DMSO). The red color represents the genes that were up-modulated; blue represents the genes that were down-regulated. B. The clustergram represents genes that are concomitantly induced by all three tested drugs.

FIG. 2 has three bar graphs, 2A, 2B, and 2C. Q-PCR analysis was performed as described in the Methods section, below, in SU-DHL-1 cells exposed to equitoxic concentrations of bendamustine, phosphoramide mustard, and chlorambucil. The levels of input cDNA were normalized using an assay for 18s RNA, and the level of transcripts in the untreated sample was set to 1. FIG. 2 A shows the relative RNA levels of two representative p53-dependent genes, p21 and NOXA. FIG. 2 B shows the RNA levels of four genes involved in the M-phase cell cycle checkpoint, polo-like-kinase 1 (PLK-1), the aurora kinases A and B, and cyclin B1. FIG. 2 C shows the relative RNA levels of genes involved in DNA-repair mechanisms, EXO1 and Fen1. The columns represents the mean+/−SE of the fold changes from DMSO-treated controls. The results were obtained from three independent experiments.

FIG. 3 shows several immunoblots that demonstrate that enhanced apoptotic effect of bendamustine (50 μM) as compared to cyclophosphamide (50 μM) and chlorambucil (4 μM) in NHL cells (SU-DHL-1). To generate these immunoblots, cell lysates were prepared after 20 hours exposure as described in the Methods section, below. Probing the membrane with β-actin served as a loading control and is shown below the regulated proteins. The top-left panel represents the expression of Ser15-phosphorylated p53, detected using a phospho-specific antibody. The middle-left panel shows total p53 and p21 expression. The lower-left panel represents the expression of Bax. The right panels shows the expression of the full-length PARP (top) and the caspase-cleaved fragment of PARP using an antibody that recognizes the specific caspase-cleavage site.

FIG. 4 consists of two graphs, A and B that represent functional analyses of selected DNA repair mechanisms. FIG. 4 A shows that bendamustine, but not cyclophosphamide, leads to DNA damage repair via base excision repair (BER). The role of the repair enzyme Ape-1, an apurininc endonuclease that plays a critical role in the BER pathway in the cytotoxic activity of bendamustine and a cyclophosphamide metabolite, phosphoramide mustard (PM), was assessed using the Ape-1 inhibitor methoxyamine (MX). The left shift of the curve observed with bendamustine and MX shows that DNA damage produced by bendamustine is repaired by BER. FIG. 4 B shows that inhibition of MGMT repair activity does not affect bendamustine cytotoxicity. The role of the repair enzyme MGMT (O⁶-methylguanine-DNA methyltransferase) in the cytotoxic activity of bendamustine was assessed using the MGMT inhibitor O⁶-benzylguanine (O⁶-BG). The addition of O⁶-benzylguanine did not significantly change the IC₅₀ of bendamustine, so it is unlikely that bendamustine induces O⁶-alkylguanine DNA adducts. In contrast, O⁶-benzylguanine significantly sensitizes cells to other nitrogen mustards such as carmustine and phosphoramide mustard (PM).

FIG. 5 illustrates that bendamustine efficiently enters tumor cells and induces prolonged and extensive DNA damage, which results in the initiation of at least three signaling pathways: 1) activation of “canonical” p53-dependent stress pathway resulting in a strong activation of intrinsic apoptosis, probably mediated by pro-apoptotic BCL-2 family members such as NOXA and Bax; 2) activation of a DNA repair mechanism, such as the base-excision repair machinery, that are not activated by other alkylating agents frequently used in NHL or CLL patients; and 3) inhibition of several mitotic checkpoints, such as the kinases PLK-1 and Aurora A and B. While not wishing to be bound to a particular theory, the concomitant induction of DNA damage and inhibition of mitotic checkpoints presumably prevents tumor cells exposed to bendamustine from efficiently repairing DNA damage before undergoing mitosis. Cells thus enter mitosis with damaged DNA, or cells that can not proceed to “conventional” p53-dependent apoptosis, will undergo death by mitotic catastrophe. This alternative programmed cell death pathway, together with the strong activation of traditional apoptosis, is believed to be why bendamustine is very effective in killing drug-resistant cancer cells in vitro, as well as in patients having chemo-refractory tumors.

FIG. 6 is a histogram that shows the results of adenylate kinase assays performed in the course of several of the “wash-out” experiments described in Example 3, below. In these experiments, SU-DHL-1 cells were treated with either 50 μM bendamustine, 20 μM phosphoramide mustard, or 2 μM chlorambucil for either 30, 60, or 90 minutes. After the timed drug incubation, the cells were washed in 1×PBS to “wash out” the particular chemotherapeutic agent and then fresh medium was added. Cells were then cultured for 48 hours, after which time adenylate kinase assays were performed on the cell supernatants. The pink bars represent zero minutes of drug (or no drug) incubation. The green bars represent 30 minute incubations, the orange bars represent 60 minute incubations, and the purple bars represent 120 minute incubations. The results plot the level of adenylate kinase activity in the supernatants versus the three drugs and a “no drug” control. Standard deviation are represented at the top of each bar on the graph.

FIG. 7, like FIG. 6, is a histogram that shows the results of adenylate kinase assays performed in the course of several of the “wash-out” experiments described in Example 3, below. The difference between the results depicted in FIGS. 6 and 7 is that the data represented in FIG. 6 concerns 48 hours of cell culture after each of the drugs was “washed out” of the culture, whereas the data in FIG. 7 concerns 72 hours of cell culture post “washing out” the particular drug.

### DETAILED DESCRIPTION OF ILLUSTRATIVE EMBODIMENTS

As those in the art will appreciate, the following description describes certain preferred embodiments of the invention in detail, and is thus only representative and does not depict the actual scope of the invention. Before describing the present invention in detail, it is understood that the invention is not limited to the particular molecules, systems, and methodologies described, as these may vary. It is also to be understood that the terminology used herein is for the purpose of describing particular embodiments only, and is not intended to limit the scope of the invention defined by the appended claims.

### Definitions

An “alkylating agent” refers to a chemotherapeutic compound that chemically modifies DNA and disrupts its function. Some alkylating agents cause formation of cross links between nucleotides on the same strand, or the complementary strand, of a double-stranded DNA molecule, while still others cause base-pair mismatching between DNA strands. Exemplary alkylating agents include bendamustine, busulfan, carboplatin, carmustine, cisplatin, chlorambucil, cyclophosphamide, dacarbazine, hexamethylmelamine, ifosphamide, lomustine, mechlorethamine, melphalan, mitotane, mytomycin, pipobroman, procarbazine, streptozocin, thiotepa, and triethylenemelamine.

An “anti-metabolite” refers to a chemotherapeutic agent that interferes with the synthesis of biomolecules, including those required for DNA synthesis (e.g., nucleosides and nucleotides) needed to synthesize DNA. Examples of anti-metabolites include capecitabine, chlorodeoxyadenosine, cytarabine (and its activated form, ara-CMP), cytosine arabinoside, dacabazine, floxuridine, fludarabine, 5-fluorouracil, gemcitabine, hydroxyurea, 6-mercaptopurine, methotrexate, pentostatin, trimetrexate, and 6-thioguanine.

An “anti-mitotic” refers to a chemotherapeutic agent that interferes with mitosis, typically through disruption of microtubule formation. Examples of anti-mitotic compounds include navelbine, paclitaxel, taxotere, vinblastine, vincristine, vindesine, and vinorelbine.

In the context of this invention, a “chemotherapeutic agent” refers to a chemical intended to destroy malignant cells and tissues. Chemotherapeutic agents include small molecules, nucleic acids (e.g., anti-sense molecules, ribozymes, small interfering RNA molecules, etc.), and proteins (e.g., antibodies, antibody fragments, cytokines, enzymes, and peptide hormones) that have anti-tumor effects when administered to a patient in order to prevent or treat a cancer or other malignancy. Chemotherapeutic agents are often divided classes based on mechanism of action, e.g., alkylating agents, anti-metabolites, and anti-mitotic agents.

The term “combination therapy” refers to a therapeutic regimen that involves the provision of at least two distinct therapies to achieve an indicated therapeutic effect. For example, a combination therapy may involve the administration of two or more chemically distinct active ingredients, for example, a fast-acting chemotherapeutic agent and a myeloprotective agent. Alternatively, a combination therapy may involve the administration of one or more chemotherapeutic agents as well as the delivery of radiation therapy and/or surgery or other techniques to either improve the quality of life of the patient or to treat the cancer. In the context of the administration of two or more chemically distinct active ingredients, it is understood that the active ingredients may be administered as part of the same composition or as different compositions. When administered as separate compositions, the compositions comprising the different active ingredients may be administered at the same or different times, by the same or different routes, using the same of different dosing regimens, all as the particular context requires and as determined by the attending physician. Similarly, when one or more chemotherapeutic agents are combined with, for example, radiation and/or surgery, the drug(s) may be delivered before or after surgery or radiation treatment.

An “intercalating agent” refers to a chemotherapeutic agent that inserts itself between adjacent base pairs in a double-stranded DNA molecule, disrupting DNA structure and interfering with DNA replication, gene transcription, and/or the binding of DNA binding proteins to DNA

“Monotherapy” refers to a treatment regimen based on the delivery of one therapeutically effective compound, whether administered as a single dose or several doses over time.

In the context of the commercialization of pharmaceuticals, the terms “promotion”, “promote”, “promoting”, and the like refer to any and all informational, persuasive, and scientific activities conducted by or on behalf of a manufacturer, distributor, or other entity involved in the discovery, research, development, and/or commercialization of the particular pharmaceutical compound, composition, or treatment regimen intended, directly or indirectly, to induce the prescription, supply, purchase, and/or use of the compound, composition, or treatment regimen. Such activities may be directed toward anyone in the in the supply and distribution chain, including, without limitation, medical professionals (e.g., physicians and nurses), pharmacists, health care administrators, insurance company or government representatives, and patients (including potential patients). In other words, the primary aim of promotion is to stimulate the sale or use of, and/or interest in, a particular pharmaceutical compound, composition, or treatment regimen, and thus any activity intended to serve this aim constitutes “promotion” of the particular pharmaceutical compound, composition, or treatment regimen.

A “patentable” composition, process, machine, or article of manufacture according to the invention means that the subject matter satisfies all statutory requirements for patentability at the time the analysis is performed. For example, with regard to novelty, non-obviousness, or the like, if later investigation reveals that one or more claims encompass one or more embodiments that would negate novelty, non-obviousness, etc., the claim(s), being limited by definition to “patentable” embodiments, specifically exclude the unpatentable embodiment(s). Also, the claims appended hereto are to be interpreted both to provide the broadest reasonable scope, as well as to preserve their validity. Furthermore, if one or more of the statutory requirements for patentability are amended or if the standards change for assessing whether a particular statutory requirement for patentability is satisfied from the time this application is filed or issues as a patent to a time the validity of one or more of the appended claims is questioned, the claims are to be interpreted in a way that (1) preserves their validity and (2) provides the broadest reasonable interpretation under the circumstances.

The term “pharmaceutically acceptable salt” refers to salts which retain the biological effectiveness and properties of the compounds of this invention and which are not biologically or otherwise undesirable. In many cases, the compounds of this invention are capable of forming acid and/or base salts by virtue of the presence of amino and/or carboxyl groups or groups similar thereto. Pharmaceutically acceptable acid addition salts may be prepared from inorganic and organic acids, while pharmaceutically acceptable base addition salts can be prepared from inorganic and organic bases. For a review of pharmaceutically acceptable salts see Berge, et al. ((1977) J. Pharm. Sci., vol. 66, 1). The expression “non-toxic pharmaceutically acceptable salts” refers to non-toxic salts formed with nontoxic, pharmaceutically acceptable inorganic or organic acids or inorganic or organic bases. For example, the salts include those derived from inorganic acids such as hydrochloric, hydrobromic, sulfuric, sulfamic, phosphoric, nitric, and the like, as well as salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicyclic, sulfanilic, fumaric, methanesulfonic, and toluenesulfonic acid and the like. Salts also include those from inorganic bases, such as ammonia, hydroxyethylamine and hydrazine. Suitable organic bases include methylamine, ethylamine, propylamine, dimethylamine, diethylamine, trimethylamine, triethylamine, ethylenediamine, hydroxyethylamine, morpholine, piperazine, and guanidine.

A “plurality” means more than one.

The term “rituximab refractory” means prior treatment with rituximab, but inappropriate for further treatment due to disease refractory to rituximab therapy, given either as a single agent or in combination (defined as no response, or progression within 6 months of completing rituximab treatment), and/or untoward reaction to prior rituximab therapy, making further treatment unwarranted, as determined by the physician or treating specialist.

The term “anti-CD20 refractory” means prior treatment with an agent that interacts with the CD20 antigen, but inappropriate for further treatment due to disease refractory to the anti-CD20 agent given either as a single agent or in combination (defined as not response, or progression within 6 months of completing the anti-CD20 treatment), and/or untoward reaction to prior anti-CD20 therapy, making further treatment unwarranted, as determined by the physician or treating specialist.

The “S phase” of the cell cycle refers to the phase in which the chromosomes are replicated.

The term “species” is used herein in various contexts, e.g., a particular species of chemotherapeutic agent. In each context, the term refers to a population of chemically indistinct molecules of the sort referred in the particular context.

A “subject” or “patient” refers to an animal in need of treatment that can be effected by molecules of the invention. Animals that can be treated in accordance with the invention include vertebrates, with mammals such as bovine, canine, equine, feline, ovine, porcine, and primate (including humans and non-humans primates) animals being particularly preferred examples.

A “therapeutically effective amount” refers to an amount of an active ingredient sufficient to effect treatment when administered to a subject in need of such treatment. In the context of cancer therapy, a “therapeutically effective amount” is one that produces an objectively measured change in one or more parameters associated with cancer cell survival or metabolism, including an increase or decrease in the expression of one or more genes correlated with the particular cancer, reduction in tumor burden, cancer cell lysis, the detection of one or more cancer cell death markers in a biological sample (e.g., a biopsy and an aliquot of a bodily fluid such as whole blood, plasma, serum, urine, etc.), induction of induction apoptosis or other cell death pathways, etc. Of course, the therapeutically effective amount will vary depending upon the particular subject and condition being treated, the weight and age of the subject, the severity of the disease condition, the particular compound chosen, the dosing regimen to be followed, timing of administration, the manner of administration and the like, all of which can readily be determined by one of ordinary skill in the art. It will be appreciated that in the context of combination therapy, what constitutes a therapeutically effective amount of a particular active ingredient may differ from what constitutes a therapeutically effective amount of the active ingredient when administered as a monotherapy (i.e., a therapeutic regimen that employs only one chemical entity as the active ingredient).

The term “treatment” or “treating” means any treatment of a disease or disorder, including preventing or protecting against the disease or disorder (that is, causing the clinical symptoms not to develop); inhibiting the disease or disorder (i.e., arresting or suppressing the development of clinical symptoms; and/or relieving the disease or disorder (i.e., causing the regression of clinical symptoms). As will be appreciated, it is not always possible to distinguish between “preventing” and “suppressing” a disease or disorder since the ultimate inductive event or events may be unknown or latent. Accordingly, the term “prophylaxis” will be understood to constitute a type of “treatment” that encompasses both “preventing” and “suppressing”. The term “protection” thus includes “prophylaxis.”

The present invention is based on the surprising discovery that the alkylating agent bendamustine exerts very rapid cytotoxic effects on a number of cancer cell types, including those refractory to conventional chemotherapeutic regimens. It has also been discovered that bendamustine exerts its toxic effects through distinct modes of action, as compared to other anti-cancer drugs, as described in detail below.

Bendamustine, 4-{5-[bis(2-chloroethyl)amino]-1-methyl-2-benzimidazolyl}, is a chemotherapeutic agent of the nitrogen mustard class. Bendamustine primarily exhibits alklyating activity, i.e., it is a DNA-damaging agent. When administered to humans (typically by bolus intravenous infusion), bendamustine has a short serum half-life, on the order of 2 hours.

Thus, it is rapidly cleared from a patient's system. Surprisingly, it has been discovered that, after cell uptake, bendamustine rapidly exerts its durable cytotoxic effects. Indeed, as reported in Example 3, below, the vast majority of the compound's cytotoxic effects are exerted upon exposing cancer cells to the agent for as little as about 30 minutes.

Current protocols for bendamustine treatment typically involve the delivery of three separate bolus intravenous infusions each containing an equivalent amount of bendamustine. The second infusion is generally given one day after the first infusion, followed by the third infusion three weeks after the first infusion. This regimen has been used due toxicities related to bendamustine treatment, including myelosuppression. Given the short serum half-life of bendamustine and its fast-acting nature, drug-related toxicity can be reduced by delaying the second and subsequent administrations. Indeed, because extensive and perhaps lethal tumor lysis has been occasionally been reported in connection with bendamustine treatment of non-Hodgkin's lymphoma, greater spacing of the multiple administrations of the drug may serve to reduce the incidence of tumor lysis. In addition to reducing unwanted toxicity, greater spacing of bendamustine administrations in a particular treatment regimen will also serve to increase the therapeutic window, i.e., the time period over which the drug is exerting its intended therapeutic benefit.

The composition(s) used in the practice of the invention may be processed in accordance with conventional methods of pharmaceutical compounding techniques to produce medicinal agents (i.e., medicaments or therapeutic compositions) for administration to subjects, including humans and other mammals, i.e., “pharmaceutical” and “veterinary” administration, respectively. See, for example, the latest edition of Remington's Pharmaceutical Sciences (Mack Publishing Co., Easton, Pa.). Typically, a compound such as bendamustine is combined as a composition with a pharmaceutically acceptable carrier. The composition(s) may also include one or more of the following: preserving agents; solubilizing agents; stabilizing agents; wetting agents; emulsifiers; sweeteners; colorants; odorants; salts; buffers; coating agents; and antioxidants.

The drugs used in the practice of the invention may be prepared as free acids or bases, which are then preferably combined with a suitable compound to yield a pharmaceutically acceptable salt. The expression “pharmaceutically acceptable salts” refers to non-toxic salts formed with nontoxic, pharmaceutically acceptable inorganic or organic acids or inorganic or organic bases. For example, the salts include those derived from inorganic acids such as hydrochloric, hydrobromic, sulfuric, sulfamic, phosphoric, nitric, and the like, as well as salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicyclic, sulfanilic, fumaric, methanesulfonic, and toluenesulfonic acid and the like. Salts also include those from inorganic bases, such as ammonia, hydroxyethylamine and hydrazine. Suitable organic bases include methylamine, ethylamine, propylamine, dimethylamine, diethylamine, trimethylamine, triethylamine, ethylenediamine, hydroxyethylamine, morpholine, piperazine, and guanidine.

In any event, the therapeutic compositions are preferably made in the form of a dosage unit containing a given amount of a desired therapeutic agent (e.g., bendamustine) and a carrier (i.e., a physiologically acceptable excipient). What constitutes a therapeutically effective amount of any such molecule for a human or other mammal (or other animal) will depend on a variety of factors, including, among others, the type of disease or disorder, the age, weight, gender, medical condition of the subject, the severity of the condition, the route of administration, and the particular compound employed. Thus, dosage regimens may vary widely, but can be determined routinely using standard methods. In any event, an “effective amount” of chemotherapeutic agent is an amount that elicits the desired cytotoxic. The quantity of such a therapeutic molecule required to achieve the desired effect will depend on numerous considerations, including the particular molecule itself, the disease or disorder to be treated, the capacity of the subject's cancer to respond to the molecule, route of administration, etc. Precise amounts of the molecule required to achieve the desired effect will depend on the judgment of the practitioner and are peculiar to each individual subject. However, suitable dosages may range from about several nanograms (ng) to about several milligrams (mg) of active ingredient per kilogram body weight per day.

The preparation of therapeutic compositions is well understood in the art. Typically, such compositions are prepared as injectable, either as liquid solutions or suspensions, however, solid forms suitable for solution in, or suspension in, liquid prior to injection can also be prepared. The preparation can also be emulsified. The active therapeutic ingredient is often mixed with excipients that are physiologically acceptable and compatible with the active ingredient. Suitable excipients are, for example, water for injection, saline, dextrose, glycerol, ethanol, or the like and combinations thereof. In addition, if desired, the composition can contain minor amounts of auxiliary substances such as wetting or emulsifying agents, anti-pyretics, stabilizing agents, thickening agents, suspending agents, anesthetics, preservatives, antioxidants, bacteriostatic agents, analgesics, pH buffering agents, etc. that enhance the effectiveness of the active ingredient. Such components can provide additional therapeutic benefit, or act towards preventing any potential side effects that may be posed as a result of administration of the pharmaceutical composition.

The compositions of the invention may be administered orally, parentally, by inhalation spray, rectally, intranodally, intrathecally, or topically in dosage unit formulations containing conventional carriers, adjuvants, and vehicles. In the context of therapeutic compositions intended for human administration, pharmaceutically acceptable carriers are used. The terms “pharmaceutically acceptable carrier” and “physiologically acceptable carrier” refer to molecular entities and compositions that are physiologically tolerable and do not typically produce an unintended allergic or similar untoward reaction, such as gastric upset, dizziness and the like, when administered to a subject.

For oral administration, the composition may be of any suitable form, including, for example, a capsule, tablet, lozenge, pastille, powder, suspension, or liquid, among others. Liquids may be administered by injection as a composition with suitable carriers including saline, dextrose, or water. The term “parenteral” includes infusion (including continuous or intermittent infusion) and injection via a subcutaneous, intravenous, intramuscular, intrasternal, or intraperitoneal route. Suppositories for rectal administration can be prepared by mixing the active ingredient(s) with a suitable non-irritating excipient such as cocoa butter and/or polyethylene glycols that are solid at ordinary temperatures but liquid at physiological temperatures.

The compositions may also be prepared in a solid form (including granules, powders or suppositories). The compositions may be subjected to conventional pharmaceutical operations such as sterilization and/or may contain conventional adjuvants, such as preservatives, stabilizers, wetting agents, emulsifiers, buffers etc. Solid dosage forms for oral administration may include capsules, tablets, pills, powders, and granules. In such solid dosage forms, the active compound may be admixed with at least one inert excipient such as sucrose, lactose, or starch. Such dosage forms may also comprise additional substances other than inert diluents, e.g., lubricating agents such as magnesium stearate. In the case of capsules, tablets, and pills, the dosage forms may also comprise buffering agents. Tablets and pills can additionally be prepared with enteric coatings. Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting sweetening, flavoring, and perfuming agents.

Injectable preparations, such as sterile injectable aqueous or oleaginous suspensions, may be formulated according to known methods using suitable dispersing or wetting agents and suspending agents. The injectable preparation may also be a sterile injectable solution or suspension in a non-toxic parenterally acceptable diluent or solvent. Suitable vehicles and solvents that may be employed are water for injection, Ringer's solution, and isotonic sodium chloride solution, among others. In addition, sterile, fixed oils can be employed as a solvent or suspending medium. For this purpose, any bland fixed oil may be employed, including synthetic mono- or diglycerides. In addition, fatty acids such as oleic acid find use in the preparation of injectables.

For topical administration, a suitable topical dose of a composition may be administered one to four, and preferably two or three, times daily. The dose may also be administered with intervening days during which no dose is applied. Suitable compositions for topical delivery often comprise from 0.001% to 10% w/w of active ingredient, for example, from 1% to 2% by weight of the formulation, although it may comprise as much as 10% w/w, but preferably not more than 5% w/w, and more preferably from 0.1% to 1% of the formulation. Formulations suitable for topical administration include liquid or semi-liquid preparations suitable for penetration through the skin (e.g., liniments, lotions, ointments, creams, or pastes), and drops suitable for administration to the eye, ear, or nose.

Exemplary methods for administering the compositions of the invention (e.g., so as to achieve sterile or aseptic conditions) will be apparent to the skilled artisan. Certain methods suitable for such purposes are set forth in Goodman and Gilman's The Pharmacological Basis of Therapeutics, 7th Ed. (1985). The administration to the patient can be intermittent; or at a gradual, continuous, constant, or controlled rate.

Typical therapeutically effective doses for bendamustine for the treatment of non-Hodgkin's lymphoma can be from about 60-120 mg/m² given as a single dose on two consecutive days, or with several days between doses. The cycle can be repeated about every three to four weeks. For the treatment of chronic lymphocytic leukemia (CLL) bendamustine can be given at about 80-100 mg/m² on days 1 and 2. The cycle can be repeated after about 4 weeks. For the treatment of Hodgkin's disease (stages II-IV), bendamustine can be given in the “DBVBe regimen” with daunorubicin 25 mg/m² on days 1 and 15, bleomycin 10 mg/m² on days 1 and 15, vincristine 1.4 mg/m² on days 1 and 15, and bendamustine 50 mg/m² on days 1-5 with repetition of the cycle about every 4 weeks. For breast cancer, bendamustine (120 mg/m²) on days 1 and 8 can be given in combination with methotrexate 40 mg/m² on days 1 and 8, and 5-fluorouracil 600 mg/m² on days 1 and 8 with repetition of the cycle about every 4 weeks. As a second-line of therapy for breast cancer, bendamustine can be given at about 100-150 mg/m² on days 1 and 2 with repetition of the cycle about every 4 weeks.

The methods of the invention involve both monotherapy and combination therapy. In the context of combination therapy, the invention envisions the administration of two or more chemotherapeutic agents. A wide variety of chemotherapeutic agents are known in the art. Some of these compounds have already been approved for use in treating one or more cancer indications. Others are in various stages of pre-clinical and clinical development. Examples of chemotherapeutic agents useful in the practice of combination therapies according to the invention include the alkylating agents busulfan, carboplatin, carmustine, cisplatin, chlorambucil, cyclophosphamide, dacarbazine, hexamethylmelamine, ifosphamide, lomustine, mechlorethamine, melphalan, mitotane, mytomycin, pipobroman, procarbazine, streptozocin, thiotepa, and triethylenemelamine. Preferred anti-metabolites for use in conjunction with bendamustine include capecitabine, chlorodeoxyadenosine, cytarabine (and its activated form, ara-CMP), cytosine arabinoside, dacabazine, floxuridine, fludarabine, 5-fluorouracil, gemcitabine, hydroxyurea, 6-mercaptopurine, methotrexate, pentostatin, trimetrexate, and 6-thioguanine. Preferred anti-mitotic compounds that can be used in combination therapies with bendamustine include navelbine, paclitaxel, taxotere, vinblastine, vincristine, vindesine, and vinorelbine.

Other classes of chemotherapeutic agents include topoisomerase I inhibitors (e.g., camptothecin, irinotecan, topotecan, etc.); topoisomerase II inhibitors such as daunorubicin, doxorubicin, etoposide, idarubicin, mitoxantrone, and teniposide; angiogenesis inhibitors (e.g., dalteparin, suramin, etc.); antibodies, including alemtuzumab, bevacizumab, bexarotene, epratuzumab, gemtuzumab ozogamicin, ibritumomab tiuxetan, imatinib mesylate, raltitrexed, revlimid, rituximab, trastuzumab; tyrosine kinase inhibitors; intercalating agents; and hormones, such as anastrozole, estrogen, anti-estrogen (e.g., fulvestrant and tamoxifen), exemestane, flutamide, goserelin, leuprolide, nilutamide, levimasole, letrozole, prednisone, and toremifene. Other chemotherapeutic agents include proteins such as angiostatin, asparaginase, deniluekin diftitox, endostatin, imiquimod, interferon, interleukin-11, and pegaspargase. Still other chemotherapeutic agents include molecules such as alitretinoin, altretamine, amifostine, amsacrine, arsenic trioxide, bleomycin, capecitabine, carboxyamidotriazole, celecoxib, dactinomycin, epirubicin, geldanmycin, 17-Allylamino-17-demethoxygeldanamycin (17 AAG), irinotecan, 2-methoxyestradiol, mithramycin, mytomycin C, oxaliplatin, squalamine, temozolamide, thalidomide, tretinoin triapine, and valrubicin. As those in the art will appreciate, these and other chemotherapeutic agents now known or later developed may be used in combination with bendamustine to treat various neoplasias, including cancers.

### EXAMPLES

The following examples are provided to illustrate certain aspects of the present invention and to aid those of skill in the art in practicing the invention. These examples are in no way to be considered to limit the scope of the invention in any manner.

### Example 1

### Molecular Analysis of the Mechanism of Action of Bendamustine

A. Introduction.

Bendamustine (Treanda™, Salmedix, Inc. CA; Ribomustin™ (Ribosepharm GmbH, Munich Germany)) is an anti-tumor agent with demonstrated preclinical and clinical activity against various human cancers, such as Non-Hodgkin's Lymphomas (NHL), chronic lymphocytic leukemias, solid tumors, breast and small cell lung cancers, and multiple myelomas, including those refractory to conventional DNA-damaging agents. Bendamustine, 4-{5-[bis(2-chloroethyl)amino]-1-methyl-2-benzimidazolyl} butyric acid hydrochloride, was originally synthesized with the intention of producing an agent with low toxicity and both alkylating and anti-metabolite properties. It has three sub-structural elements: a 2-chloroethylamine alkylating group; a benzimidazole ring; and a butyric acid side-chain. The 2-chloroethylamine alkylating group is shared with other nitrogen mustards, such as cyclophosphamide, chlorambucil, and melphalan. The benzimidazole central ring system is a unique feature of bendamustine, although the butyric acid side chain is present in chlorambucil. This multi-faceted structure may contribute to its unique anti-neoplastic activity profile and distinguishes it from conventional alkylating agents.

DNA alkylating agents are extremely useful in the chemotherapy armamentarium. Such drugs may possess unexpected mechanisms of action, such as a capacity of some of these compounds to induce programmed necrosis and the capacity of others (e.g., platins) to induce apoptosis even in cells deprived of nuclei. In the case of the “nitrogen mustards”, major differences exist in their profile of activity as reflected by their differentiated use in various indications: cyclophosphamide, which is used primarily in treating NHL; chlorambucil, which is used in treating chronic lymphocytic leukemia; and melphalan, which is used in treating multiple myeloma.

The main anti-tumor action of bendamustine, in common with other alkylating agents, results from the formation of cross-links between the paired strands of DNA, although other modes of action may also be involved. Thus, the anti-tumor action of bendamustine may derive from mechanisms which are more complex than simply classic alkylation activity, as DNA double-strand breaks caused by bendamustine are significantly more durable than those caused by cyclophosphamide or BNCU, bendamustine shows activity against cell lines which are resistant in vitro and ex vivo to other alkylating agents, and unique pro-apoptotic activity has been demonstrated by bendamustine as a single agent and in combination with other anti-cancer agents in several in vitro tumor models. Detailed molecular studies on the exact mechanism of action of bendamustine remain sparse. For this reason, state-of-the art molecular tools were used to fully dissect the mechanism of action of bendamustine. This example presents results derived from pharmacogenomic assays to analyze the gene expression profile changes induced by bendamustine in NHL cell lines. These pharmacogenomic analyses were validated by functional assays dealing with the initiation of apoptotic signaling, the mechanism of DNA repair, and the modulation of mitotic checkpoints. Finally, bendamustine has been profiled in the National Cancer Institute's human tumor 60 cell line in vitro screen, and its comparative activity against a library of other alkylating agents (i.e., chlorambucil and phosphoramide mustard (the metabolite of cyclophosphamide)) was studied. Results were also generated using pharmacogenomic assays to analyze the gene expression profile changes induced by bendamustine in NHL cell lines. These pharmacogenomic analyses were validated by Q-PCR and functional assays dealing with the initiation of apoptotic signaling, mechanisms of DNA repair, and the modulation of mitotic checkpoints. Together, these results demonstrate that bendamustine possesses multiple mechanisms of action that are distinct from other DNA alkylating drugs, explaining bendamustine's activity in patients having tumors refractory to conventional therapy.

B. Materials and Methods.

a. Cells.

SU-DHL-1 cells were obtained from the University California San Diego. Cells were grown in RPMI 1640 (Hyclone) supplemented with 10% FBS (Invitrogen) and 100 units/ml penicillin/streptomycin.

b. Reagents.

Bendamustine hydrochloride was obtained from Fujisawa Deutschland (Munich, Germany). Phosphoramide mustard cyclohexylamine salt (PM, NSC69945), an active metabolite of cyclophosphamide, was obtained from the synthetic repository of the Developmental Therapeutics Program (DTP) at the National Cancer Institute (NCI). All other reagents were obtained from commercial sources such as Sigma-Aldrich.

c. Drug Treatments.

For most of the assays presented in this example, the concentrations used for bendamustine, phosphoramide mustard (the active metabolite of cyclophosphamide), and chlorambucil were selected based on their cytotoxic activity measured with the MTT assay over a period of three days. Drugs were prepared in DMSO and then diluted in culture medium.

d. Preparation of RNA Samples and Analysis of Expression Data.

Cells were harvested (5×10⁶ cells) in 1 mL TRIZOL solution (Invitrogen, San Diego, Calif.) and total RNA was isolated as per manufacturer's instructions. Biotin-labeled cDNA (15 μg) was hybridized to each GeneChip array (Affymetrix, Santa Clara). Briefly, the procedure to prepare material for hybridization to the chips involved multiple steps. Total RNA was isolated and quantified by optical density. cDNA was generated using a specific primer that recognizes the poly A tail coupled with a T7 promoter (dT7-(T)24) with dNTP, DTT, and Superscript II to generate the first strand cDNA. This approach alleviated the need to isolate poly-A(+) mRNA. The second strand was synthesized by adding dNTPs with DNA ligase, DNA pol I, and RNAse H, and incubating for 2 h at 16° C. before adding T4 DNA polymerase for an additional 5 min. cDNA was column purified and quantified. In vitro transcription (IVT) was performed prior to hybridization to the high-density oligonucleotide arrays. The starting material for this reaction was 1 μg of cDNA to which NTPs were added with 25% less CTP and UTP to be compensated by adding 10 mM biotinylated-11-CTP and 10 mM biotinylated-16-UTP. The final addition of T7 enzyme in the appropriate buffer for 6 h at 37° C. yielded the biotinylated IVT RNA which was then column purified (RNeasy, Qiagen). Chemically fragmented IVT RNA (15 μg) was mixed with control oligonucleotides, standards (including a housekeeping gene), and salmon sperm DNA in the appropriate buffer, heated to 95° C. for 5 minutes, and hybridized to the chip for 16 h at 42° C. Non-hybridized material was washed off with 2×SSPE and phycoerythrin-labeled avidin was then added to the reaction. The excess fluorochrome was washed off and the chip was then scanned for intensity of fluorescence in each synthesis feature (synthesis features are 7.5 square microns).

e. Bioinformatics Analysis.

A strategy and a process for the analysis of gene expression data was developed, which involved the use of the CORGON method to analyze scanned images of Affymetrix GeneChips. CORGON is freely available software, whose core statistical method is known (Sasik, et al. (2002), Bioinformatics, vol. 18, no. 12:1633-40). Only genes that were present at p<0.05 (95% confidence level) in at least one of the conditions were considered for further analysis. A comparison of CORGON with the Affymetrix Microarray Suite (AMS) 5.0 software revealed a 4.4% false positive error rate for CORGON as compared to 29% for AMS 5.0. The genes selected were sorted according to the average or peak magnitude of modulation. The top 100 most modulated genes were chosen for clustering based on the similarity of their expression pattern. Hierarchical clustering methods were used. This initial classification was extremely useful in determining what were the primary genes and pathways modulated by the process under investigation. Clusters of genes that appeared to be co-regulated were subjected to promoter analysis. The next step was GO3 analysis, an unbiased and unsupervised tool for finding statistically significant terms in the Gene Ontology database (website: www.geneontology.org) related to the process. GO3 facilitates the process of identifying the critical components of the system that were modulated significantly. There were three ontologies in the database: molecular function; biological process; and cellular component. The analysis was performed at the UCSD Center for AIDS Research Genomics Core Facility.

f. Quantitative PCR Analysis.

The expression levels of specific transcripts were determined using quantitative PCR (Q-PCR). Total RNA from each treated SU-DHL-1 cell pellet was isolated using an RNeasy mini-prep kit (Qiagen, Valencia, Calif.). cDNAs were made using a ThermoScript reverse-transcriptase kit (Invitrogen) and oligo-dT primers according to the manufacturer's protocol. Q-PCR amplification and quantitation was carried out using an iCycler machine (Bio-RAD, Hercules, Calif.). Sample amplification was performed in a volume of 25 μL containing 12.5 μL of 2×IQ SybrGreen™ Mix (Bio-Rad), 1 μM of each primer, and a volume of cDNA corresponding to 80 ng of total RNA. Cycling conditions were: 95° C. for 5 seconds; 30 seconds at the appropriate annealing temperature for each primer; and 72° C. for 30 seconds. Target specificity of the assays was validated by melt curve analysis. The expression of each gene was normalized relative to 18s expression levels for each sample. The expression of each gene relative to untreated control was then calculated per the method of Livak and Schmittgen ((2001), Methods, vol. 25:402-408). Primers were designed using Beacon Designer™ (Premier Biosoft, Palo Alto, Calif.) or designed based on the literature. Primer sequences and annealing temperatures are as follows (each primer is written 5′ to 3′, followed by its SEQ ID NO):

| | | | |
|---|---|---|---|
| | | | Anneal |
| Gene ID | Forward Primer | Reverse Primer | Temp |

| | | | |
|---|---|---|---|
| 18s | CGCCGCTAGAGGTGAAATTC | TTGGCAAATGCTTTCGCT | 55° C. |
| | (SEQ ID NO. 1) | (SEQ ID NO. 2) | |
| p21 | CCTCATCCCGTGTTCTCCTTT | GTACCACCCAGCGGACAAGT | 57° C. |
| | (SEQ ID NO. 3) | (SEQ ID NO. 4) | |
| Noxa | ATTTCTTCGGTCACTACACAA | AACGCCCAACAGGAACAC | 55° C. |
| | (SEQ ID NO. 5) | (SEQ ID NO. 6) | |
| PLK-1 | CTCAACACGCCTCATCCT | GTGCTCGCTCATGTAATTGC | 57° C. |
| | (SEQ ID NO. 7) | (SEQ ID NO. 8) | |
| Aurora A | TCCTTGTCAGAATCCATTACCTGT | GAATGCGCTGGGAAGAATTTG | 55° C. |
| | (SEQ ID NO. 9) | (SEQ ID NO. 10) | |
| Aurora B | AGAGTGCATCACACAACGAGA | CTGAGCAGTTTGGAGATGAGGTC | 56° C. |
| | (SEQ ID NO. 11) | (SEQ ID NO. 12) | |
| Cyclin B1 | AGTGTGACCCAGACTGCCTC | CAAGCCAGGTCCACCTCCTC | 57° C. |
| | (SEQ ID NO. 13) | (SEQ ID NO. 14) | |
| Exo1 | TTGGTCTGGAGGTCTTGGAGA | GAATCGCTCTTTCTTCGGAACTG | 57° C. |
| | (SEQ ID NO. 15) | (SEQ ID NO. 16) | |

g. COMPARE Analysis.

Bendamustine was tested in the NCI's in vitro anti-tumor screen consisting of 60 human tumor cell lines. Testing involved a minimum of five concentrations at 10-fold dilutions, and each screen was repeated twice. A 48 hour continuous drug exposure protocol was used. A Sulforhodamine B protein assay estimated cell viability or growth. The COMPARE method and associated data are freely available on the Developmental Therapeutics Program (DTP) website (website: dtp.nci.nih.gov). The NCI assigned bendamustine the number: NSC138783.

h. Western Blot Analysis.

SU-DHL-1 cells were incubated with 50 μM bendamustine, 2 μM chlorambucil, or 20 μM phosphoramide mustard for 20 hours. Cells were washed twice with 1×PBS and lysed for 1 hour with ice cold lysis buffer (1 M Tris-HCl (pH 7.4), 1 M KCl, 5 mM EDTA, 1% NP-40, 0.5% sodium deoxycholine, with 1 mM sodium orthovanidate, 1 mM sodium fluoride, protease inhibitor cocktail (Roche, Nutley, N.J.), and phosphatase inhibitor cocktail (Sigma, St. Louis, Mo.)) added directly before lysis. Non-soluble membranes, DNA, and other precipitants were pelleted and the protein supernatant obtained. Protein concentrations were determined using the Bradford assay (Pierce, Rockford, Ill.). 20 μg of lysate were separated by gel electrophoresis on a 4-12% polyacrylamide gel, transferred to nitrocellulose membranes (Invitrogen), and detected by immunoblotting using the following primary monoclonal antibodies: anti-p53, anti-phosphorylated p53 (Ser15-specific), anti-p21, and anti-cleaved PARP (caspase-specific cleavage site), which were all purchased from Cell Signaling (Beverly, Mass.); anti-Bax and anti-PARP, which were purchased from BD Pharmingen (San Diego, Calif.), and anti-beta-actin, used for a loading control, which was purchased from Sigma (St. Louis, Mo.). Primary antibodies were incubated overnight at 4° C. with gentle shaking. Membranes were washed three times with 1×PBS and incubated with Alexa Flour 680 goat anti-mouse secondary antibody (1:4000) (Molecular Probes, Eugene, Oreg.) for 2 hours at room temperature with gentle shaking. Blots were washed three times with 1×PBS and scanned on a LiCor Odyssey scanner.

i. In Vitro Cell Based Ape-1 and AGT Assays.

Cells were pre-incubated for 30 minutes with either 6 mM methoxyamine (Sigma) or 50 μM O⁶-benzylguanine (Sigma), inhibitors of Ape-1 base excision repair enzyme and alkylguanyl transferase (AGT) enzyme, respectively. The cells were then exposed to various concentrations of the indicated agents for 72 hrs. Cytotoxicity was evaluated by the MTT assay (13) and an IC₅₀ was measured as the drug concentration that inhibited by 50% the value of the untreated control. Analyses were performed using GraphPad Prism version 3.00 GraphPad Software (San Diego, Calif.).

j. Cell Cycle Analyses.

SU-DHL-1 cells were incubated with equitoxic (IC₅₀) concentrations of bendamustine (50 μM), chlorambucil (4 μM), or phosphoramide mustard (50 μM) for 8 hours. Cells were washed with PBS and fixed in 70% ethanol 20° C. for at least one hour. Fixed cells were re-hydrated by washing with PBS. Cells were resuspended in a propidium iodide staining solution consisting of 10 μg/ml propidium iodide (Calbiochem, La Jolla, Calif.), 10 μg/ml RNAse A (DNase free, Novagen, Madison, Wis.), and 10 μl/ml Triton-X (Sigma) in PBS. Samples were analyzed using a FACSCalibur (BD Biosciences, San Jose, Calif.). Analyses of cell cycle distribution were performed using DNA ModFit LT (Verity House Software, Inc. Sunnyvale, Calif.) modeling software.

k. H2AX Foci Formation.

Cell were grown on Lab-Tek chamber slides (Nalge Nunc Intl., Naperville, Ill.) in RPMI 1640 media supplemented with 10% FBS. After allowing the cells to attach for at least one day, cells were treated in media with either DMSO or 50 μM bendamustine. The cells were incubated for 30 minutes at 37° C. and then washed two times with PBS. They were incubated for an additional 4 hours at 37° C. The cells were then washed twice with 1×PBS and incubated 10 minutes in −20° C. 100% methanol to fix the cells. They were then washed three times for five minutes each with 1×PBS. They were incubated at room temperature for 1 hour in blocking buffer (10% FBS in 1×PBS, 1% BSA). The slides were incubated at 4° C. with rocking overnight with the primary polyclonal anti-H2AX antibody (R & D Systems, Minneapolis, Minn.). The antibody was diluted in blocking buffer at a ratio of 1:10,000. Slides were washed three times with 1×PBS and incubated with Alexa Flour 488 goat anti-rabbit secondary antibody (1:4000) (Molecular Probes, Eugene, Oreg.) for 45 minutes at room temperature with gentle shaking. Slides were washed three times with 1×PBS and then the chambers removed and SlowFade Light Antifade with DAPI (Molecular Probes) was added to the cells and coverslips sealed on the slides. Analysis was performed using a motorized Zeiss AxioPlan 2e imaging microscope with DIC optics and fluorescence, a Zeiss AxioCam HRm camera and Zeiss Axiovision software Version 4.2.

l. Phosphorylation of H2AX at Residue Ser139 Immunoblot.

Cell lines were grown to confluency in RPMI 1640 media supplemented with 10% FBS. The cells were then washed twice with 1×PBS and lysed for 1 hour with ice cold lysis buffer (1 M Tris-HCl (pH 7.4), 1 M KCl, 5 mM EDTA, 1% NP-40, 0.5% sodium deoxycholine, with 1 mM sodium orthovanidate, 1 mM NaF, protease inhibitor cocktail (Roche, Nutley, N.J.), and phosphatase inhibitor cocktail (Sigma, St. Louis, Mo.)) added directly before lysis. Non-soluble membranes, DNA, and other precipitants were pelleted and the protein supernatant obtained. Protein concentrations were determined using the Bradford assay (Pierce, Rockford, Ill.). Twenty micrograms of lysate were separated by gel electrophoresis on a 4-12% polyacrylamide gel, transferred to nitrocellulose membranes (Invitrogen, Carlsbad, Calif.), and detected by immunoblotting using a polyclonal anti-H2AX antibody (R & D Systems, Minneapolis, Minn.). The antibody was diluted in blocking buffer at a ratio of 1:2000, and the membranes were incubated for 2 hours at room temperature with gentle shaking. Membranes were washed three times with 1×PBS and incubated with Alexa Flour 680 goat anti-rabbit secondary antibody (1:5000) (Molecular Probes, Eugene, Oreg.) for 2 hours at room temperature with gentle shaking. Blots were washed three times with 1×PBS and scanned on a LiCor Odyssey scanner.

C. Results.

a. Gene Expression Profiling Identifies Signature Genes that are Regulated by Bendamustine that are Distinct from Chlorambucil or Cyclophosphamide.

Equitoxic concentrations for bendamustine, chlorambucil, and phosphoramide mustard (the active metabolite of cyclophosphamide) were determined by measuring cell viability after three days exposure to drug. For the assays presented in this study, the concentrations used for bendamustine, phosphoramide mustard, and chlorambucil were selected based on this data (Table 1, below). These concentrations also reflect the clinically achievable levels for each drug. Affymetrix GeneChip analysis was used to compare the expression levels of over 12,000 genes in drug-treated SU-DHL-1, a non-Hodgkin's lymphoma cell line, cells compared to control cells. SU-DHL-1 cells were incubated with bendamustine at the IC₅₀ concentration (25 μM) and at the IC₉₀ concentration (35 μM). Chlorambucil and the cyclophosphamide metabolite phosphoramide mustard were tested at IC₉₀, i.e., 5 μM and 50 μM, respectively. Gene expression was monitored following 8 hours treatment with drug to identify the proximal events of this early stress response.

TABLE 1
IC50s of Bendamustine, PM, and
Chlorambucil in SU-DHL-1 cells

| | | Ave IC50 | | Ave IC90 | |
|---|---|---|---|---|---|
| Cell Line | Drug | (μm) | STDV | (μM) | STDV |
| SU-DHL-1 | Bendamustine | 33.2 | 10.6 | 56.3 | 16.1 |
| | Chlorambucil | 3.4 | 1.1 | 6.2 | 1.3 |
| | Phosporamide | 21.3 | 7.6 | 33.0 | 6.2 |
| | Mustard | | | | |

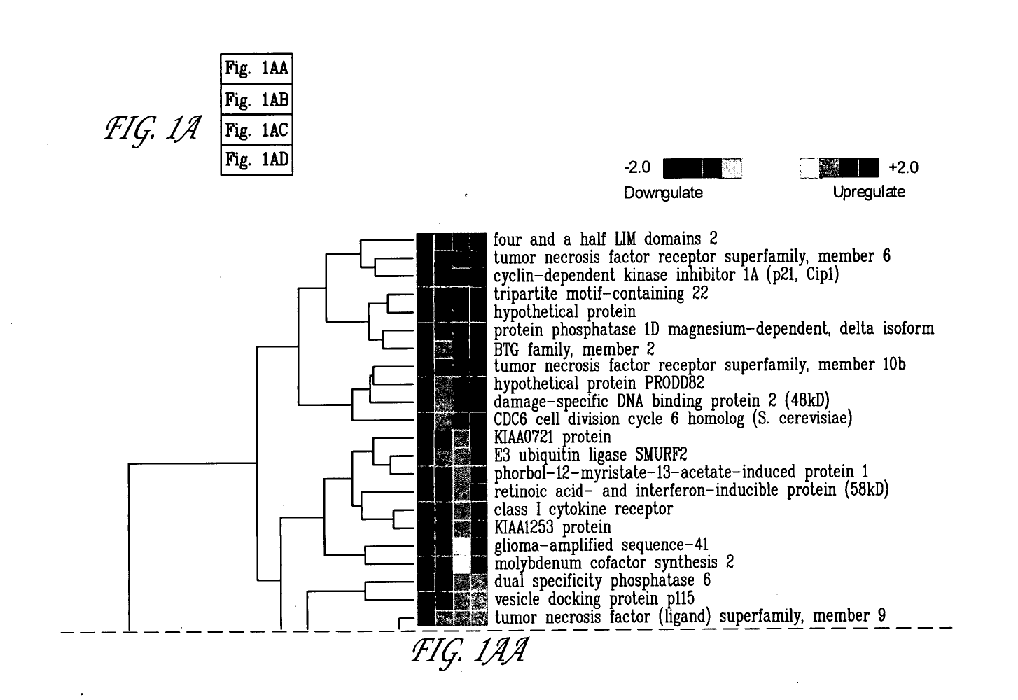

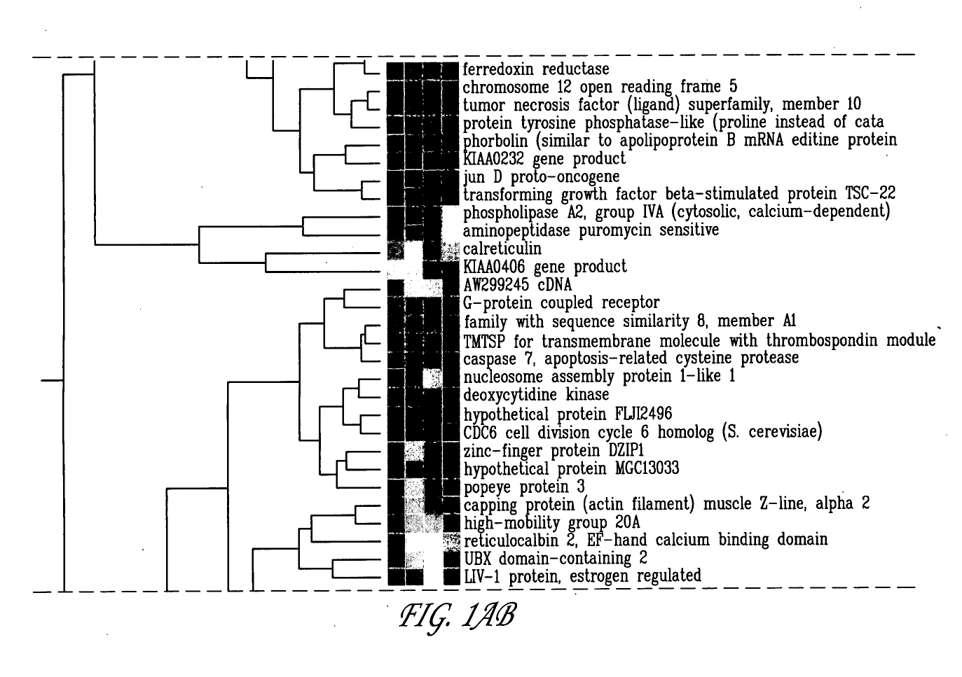

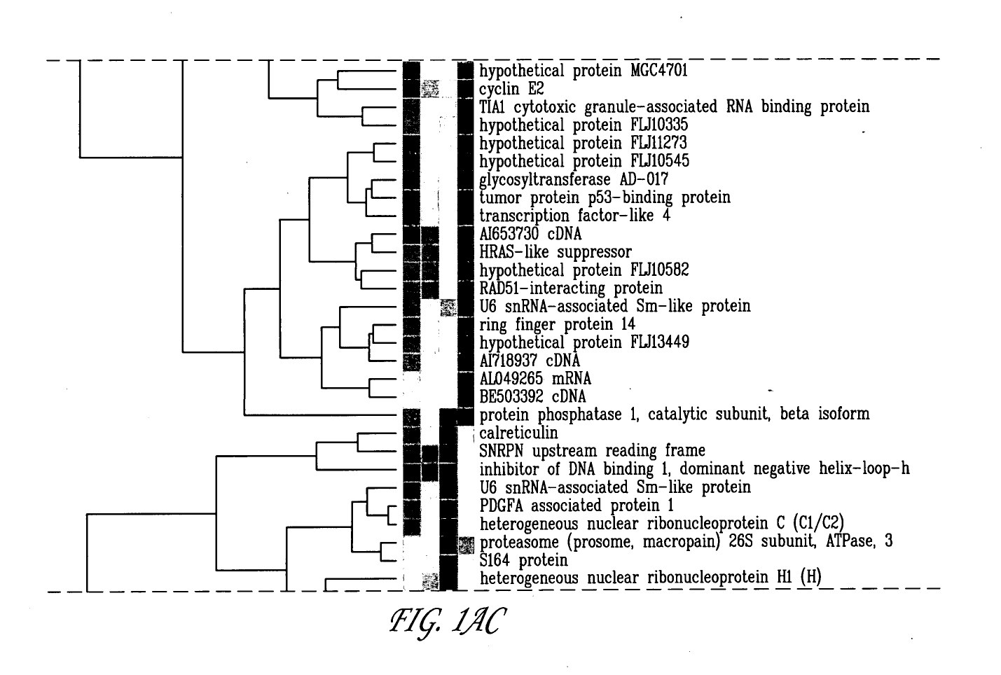

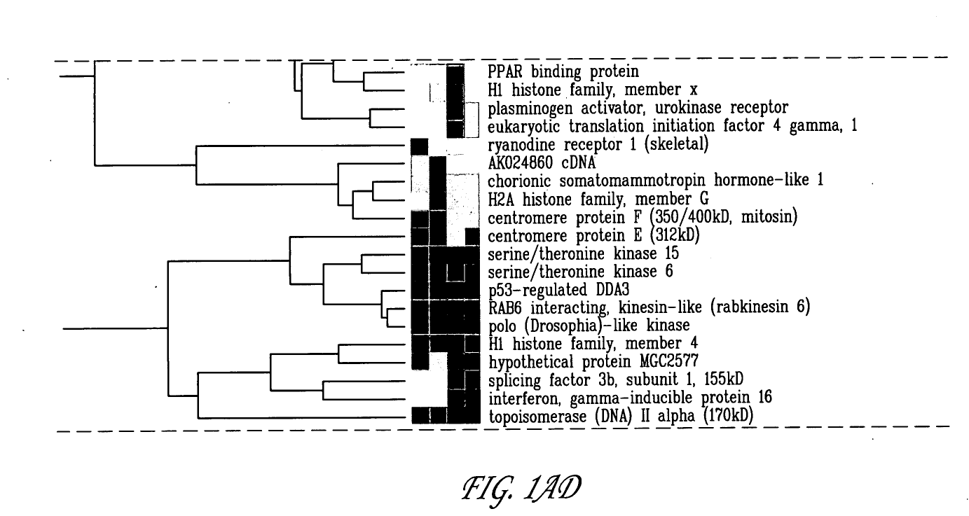

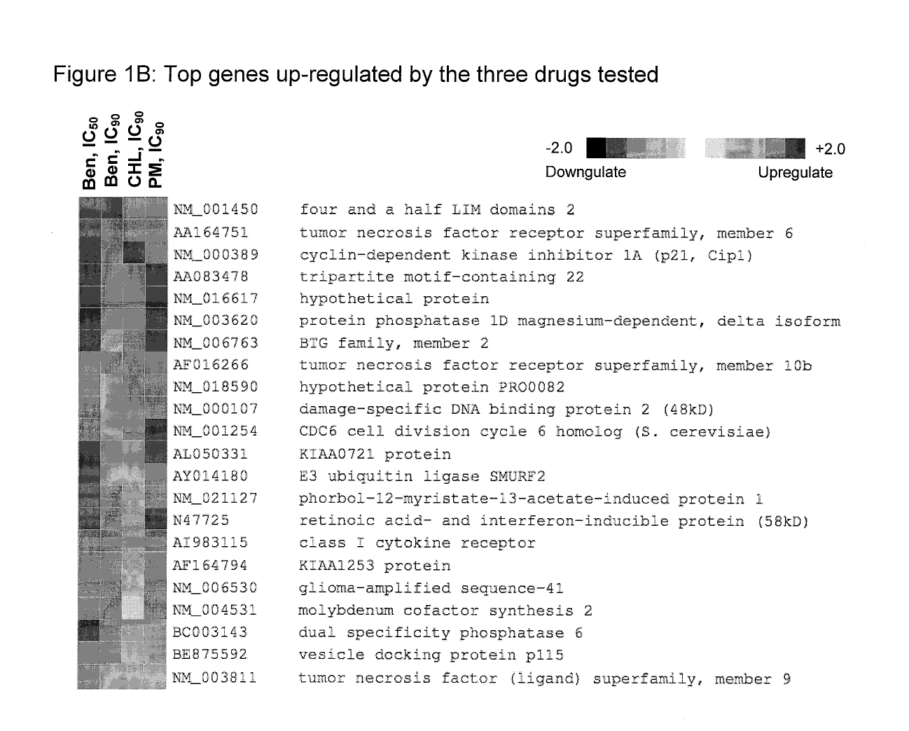

The genomic analysis revealed that the majority of the genes are similarly regulated between the three tested drugs, as demonstrated by the clustergram of the top 100 modulated genes (FIG. 1A). Most genes were upregulated (red color) upon exposure to the drugs. A subset of genes was transcriptionally repressed following drug treatment (blue color). Importantly, a group of genes was identified that displayed differential regulation by bendamustine compared to the other two drugs tested.

Many of the induced genes (FIG. 1B) were known to possess p53-response elements in their promoter regions and are considered p53-dependent. Examples of these genes are: p21 (p53-induced cell division kinase inhibitor); wip1 (p53-induced protein phosphatase 1); NOXA (p53-induced pro-apoptotic Bcl-2 family member); DR5/KILLER (p53-regulated DNA damage-inducible cell death receptor); and BTG2. Interestingly, four members of the tumor necrosis factor receptor superfamily (members 6, 9, 10, and 10b) were identified in the top-100 modulated genes. Several of these genes have been shown to play a critical role in the regulation of the extrinsic apoptotic pathway (REF, TRAIL/TNF apoptosis). Several other genes display an opposite trend between bendamustine and the other two compounds (data not shown). These genes were upregulated by bendamustine, at both concentrations, but were down-regulated by both chlorambucil and phosphoramide mustard.

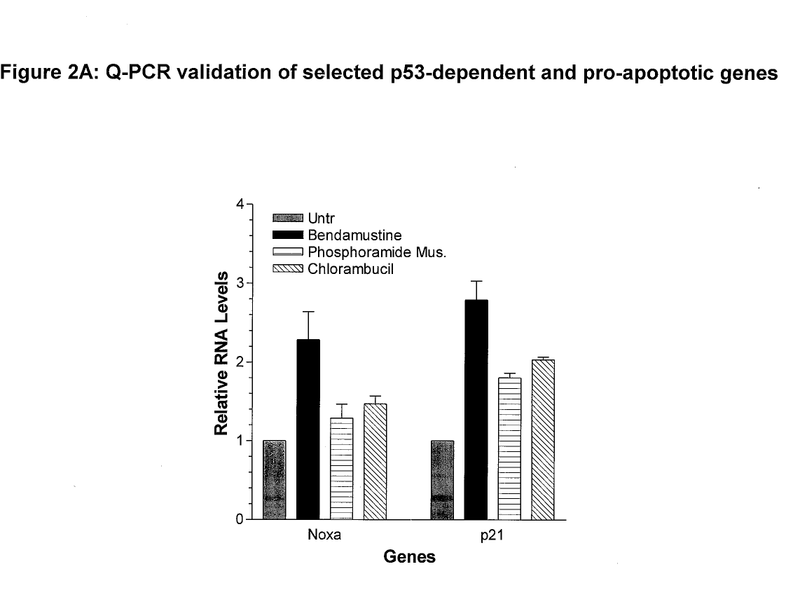

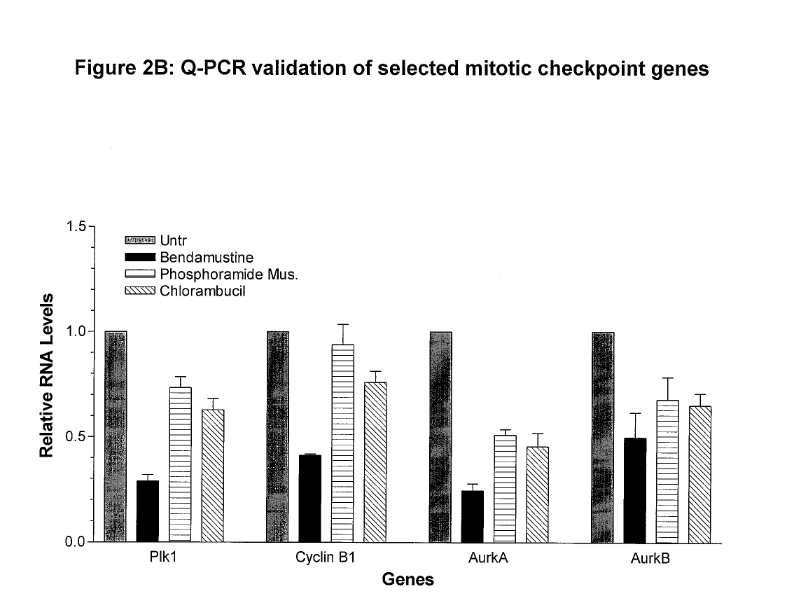

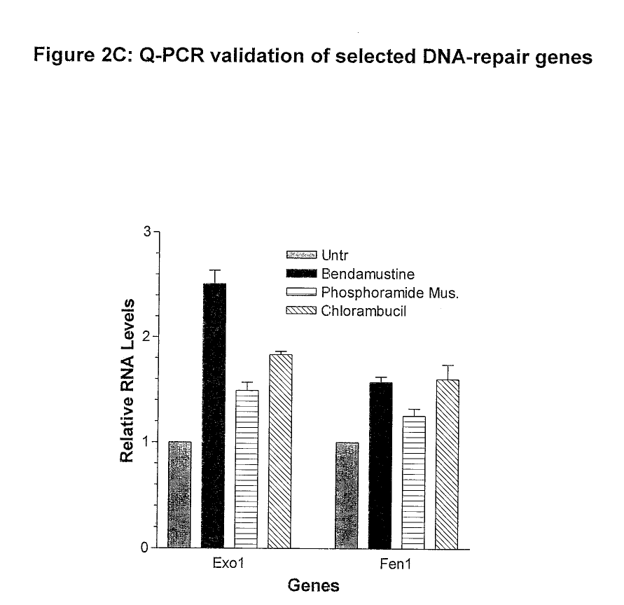

To assess the pharmacogenomic differences between bendamustine, chlorambucil, and phosphoramide mustard, the results from the gene profiling were re-analyzed with the GO3 software, an unbiased and unsupervised tool for finding statistically significant terms in the Gene Ontology (GO) database (website: www.geneontology.org) related to the process. Genes significantly up- or down-regulated in bendamustine-treated cells and at least 1.5-fold above or below levels of expression in control-treated cells were connected to biological process annotations provided by the Gene Ontology (GO) consortium. Based on the hierarchical structure of the GO annotations, the probability that each immediate daughter term (a P value) be linked to the number of selected genes by chance was calculated. The results of the GO analysis comparing the DMSO-treated control and the bendamustine-treated cells (at IC₉₀ dose) are reported in Table 2, below. See also FIG. 2C. In Table 2, below, the first column represents general categories, the second and third columns are the number and name of the specific biological process, and the last column is the p value for each process. The p value was calculated using the GO3 software. Four major functional groups were found be statistically modulated by bendamustine: (1) DNA-damage, stress response, apoptosis; (2) DNA metabolism, DNA repair, transcription; (3) cell proliferation, cell cycle, mitotic checkpoint; and (4) cell regulation. Each of these groups encompasses several biological processes that were found to be significantly modulated by bendamustine. The biological processes that provided the lowest p values and therefore were the most statistically significant were: response to DNA damage stress (GO6974); DNA metabolism (GO6259); and cell proliferation (GO8283).

TABLE 2
Results from GO-clustering analysis from bendamustine-induced
gene changes in SU-DHL-1 cells¹

| | GO | | |
|---|---|---|---|
| Functional Groups | number | GO Description: Biological Process | P value |
| DNA-damage, stress | 6974 | Response to DNA damage stress | 0.00001 |
| response, apoptosis | 6950 | Response to stress | 0.0003 |
| | 16265 | Death | 0.0482 |
| DNA metabolism, | 6259 | DNA metabolism | 0.00003 |
| DNA repair, | 6139 | Nucleobase, nucleoside, nucleotide | 0.0004 |
| transcription | | and nucleic acid | |
| | 6357 | Regulation of transcription from | 0.0003 |
| | | Pol II promoter | |
| | 6366 | Transcription from Pol II promoter | 0.0068 |
| Cell proliferation, | 8283 | Cell proliferation | 0.00001 |
| cell cycle, mitotic | 8151 | Cell growth and/or maintenance | 0.0041 |
| checkpoint | 6275 | Regulation of DNA replication | 0.0101 |
| | 278 | Mitotic cell cycle | 0.0334 |
| | 79 | Regulation of CDK activity | 0.0192 |
| | 7078 | Mitotic metaphase plate congression | 0.0470 |
| | 50790 | Regulation of enzyme activity | 0.0363 |
| Cell regulation | 50789 | Regulation of biological process | 0.00004 |
| | 50794 | Regulation of cellular process | 0.0035 |
| | 9987 | Cellular process | 0.0379 |

¹GO clustering analysis performed as described in Methods section. The table represents the terms identified from the Gene Ontology database (http://www.geneontology.org/) that are the most statistically-significantly modulated between untreated control and SU-DHL-1 treated with IC50 dose of bendamustine.

A similar analysis performed with chlorambucil and phosphoramide mustard suggested that little overlap exists between the profile obtained with bendamustine and chlorambucil. Some similarities in gene modulation were observed between bendamustine and phosphoramide mustard, although these were limited to the “DNA metabolism, DNA repair, and transcription” group. These results provided the basis for the selection of specific gene products for the quantitative validation of the gene array results and more definitive differentiation of bendamustine.

b. Validation of Genomic Analysis by Real-Time Quantitative Q-PCR Analysis.

Confirmation and validation of the array data was performed by real-time quantitative PCR analysis (Q-PCR). Several genes involved in p53-signaling, apoptosis, DNA repair, and cell cycle/mitotic checkpoints were all differentially regulated when comparing bendamustine to the other alkylating agents tested.

Two examples of “canonical” p53-dependent genes selected for Q-PCR validation were p21 (Cip1/Waf1), the cyclin-dependent kinase inhibitor 1A, and the pro-apoptotic BH3-only Bcl-2 family member, NOXA. Both genes were found to be induced in SU-DHL-1 cells, 8 hours after exposure to bendamustine. Both genes were also induced by equitoxic concentrations of phosphoramide mustard and chlorambucil, but to a much lower extent (FIG. 2A).

One of the most striking results that emerged from the validation analysis was the differential regulation of several mitosis-related genes, including polo-like kinase 1 (PLK-1), the Aurora Kinases A and B, and cyclin B1. These genes are considered to play an important in mitotic checkpoint regulation. Treatment with bendamustine led to a 60 to 80% down-regulation of the mRNA expression of all these genes. In contrast, phosphoramide mustard or chlorambucil only exerted a minor effect on the transcripts of these genes, with possibly the exception of the Aurora kinases (FIG. 2B).

Differences also emerged in the analysis of the mRNA expression of the DNA-repair gene exonuclease-1 (EXO1). Bendamustine induced a slightly stronger (2.5-fold) up-regulation of Exo1 expression (FIG. 2C) compared with that observed with phosphoramide mustard (1.5-fold) or chlorambucil (1.8-fold). Fen1 (flap endonuclease 1) was also upregulated by bendamustine, and phosphoramide mustard upregulated this gene to the same level when used at equitoxic concentrations (FIG. 2C).

c. Apoptosis Signaling by Bendamustine in NHL Cells.

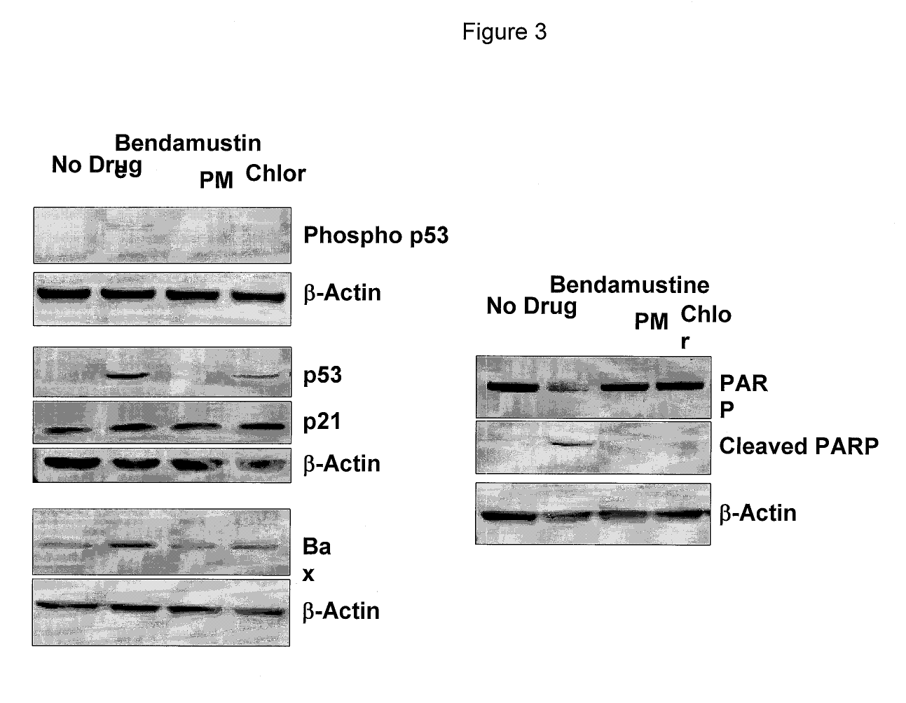

To dissect the molecular events involved in bendamustine-induced programmed cell death in NHL cells, expression of key apoptotic proteins was monitored by immunoblot analysis. The results clearly showed that bendamustine can efficiently and rapidly trigger the classical p53-dependent apoptotic pathway. One of the initial or apical events is the induction of p53 phosphorylation, as detected using antibodies that specifically recognize phosphorylation of the serine-15 residue. An 8-fold up-regulation of Ser-15-phosphorylated p53 was observed in SU-DHL-1 cells exposed to bendamustine, while only a minor up-regulation was seen in phosphoramide mustard treated cells, and no changes were observed in chlorambucil-treated cells (FIG. 3, top-left panel).

In parallel with the induction of phosphorylated p53, a strong increase in the expression of total p53 was seen in bendamustine-treated cells. Chlorambucil-treated cells displayed a small increase in total p53, while exposure to phosphoramide mustard induced no change in p53 levels. The changes observed in p21 protein expression were minor for each of the drugs when compared to changes in protein expression levels of p53. An increase in the protein expression of Bax, a key BH3-only pro-apoptotic Bcl-2 family member, was observed only in bendamustine-treated SU-DHL-1 cells (FIG. 3, low-left panel).

The most striking difference observed in comparing the effect of bendamustine with phosphoramide mustard and chlorambucil was found when the expression of PARP, poly-ADP-ribose polymerase-1, was compared. PARP is a critical NAD-requiring enzyme important in DNA-repair mechanisms. PARP is also an “early” substrate of the pro-apoptotic proteolytic caspase enzymes. SU-DHL-1 cells treated with bendamustine showed a dramatic reduction of PARP protein expression (FIG. 3, top-right panel). The reason for the reduction of PARP expression was its cleavage by caspases, as demonstrated by the appearance of proteolytic cleavage products recognized by a “cleavage-specific” antibody (FIG. 3, middle-right panel). Notably, no changes in the expression of PARP were detected in NHL cells treated by equitoxic concentrations of phosphoramide mustard or chlorambucil. Similar results were observed when using double the equitoxic doses of phosphoramide mustard (40 μM) and chlorambucil (4 μM) while maintaining the dose of bendamustine (50 μM) (data not shown). Thus, an assessment of PARP expression levels can be used for various purposes. For example, a PARP assay can be to provide an indication as to the efficacy of a particular therapeutic regimen, wherein reduced PARP expression (preferably measured at the protein level, for example by PARP activity, for the presence of PARP cleavage products, etc.) indicates that the administered drug is having the desired effect. In addition, a PARP assay can be used prognostically to determine, for example, if cells of a tissue (for example, cells derived from a biopsy or other biological sample) are likely to respond to a particular therapy (e.g., bendamustine monotherapy or a combination therapy wherein one of the therapies utilizes bendamustine).

d. Inhibition of Base Excision Repair, but not O⁶-methylguanine-DNA Methyltransferase Repair, Blocks Bendamustine Activity.

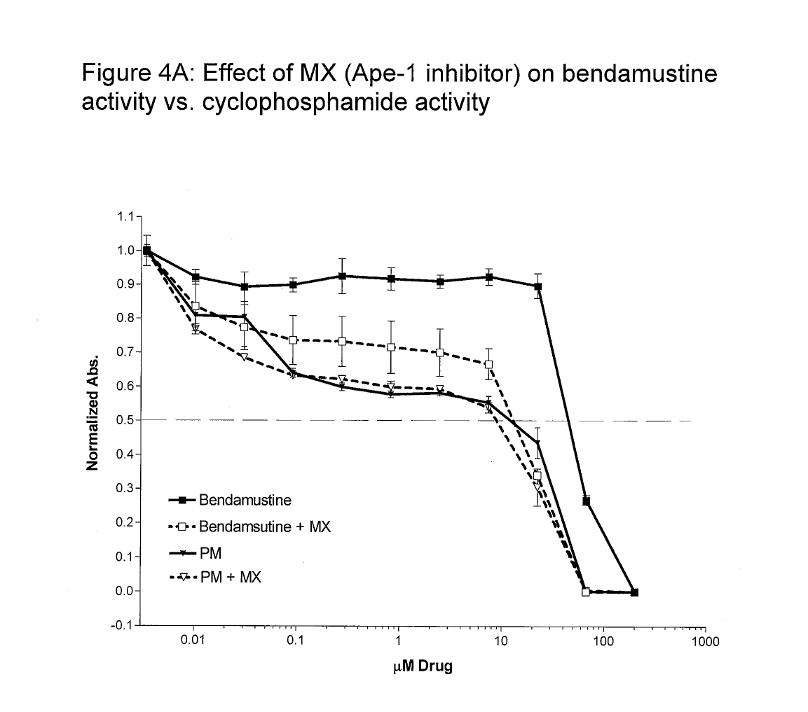

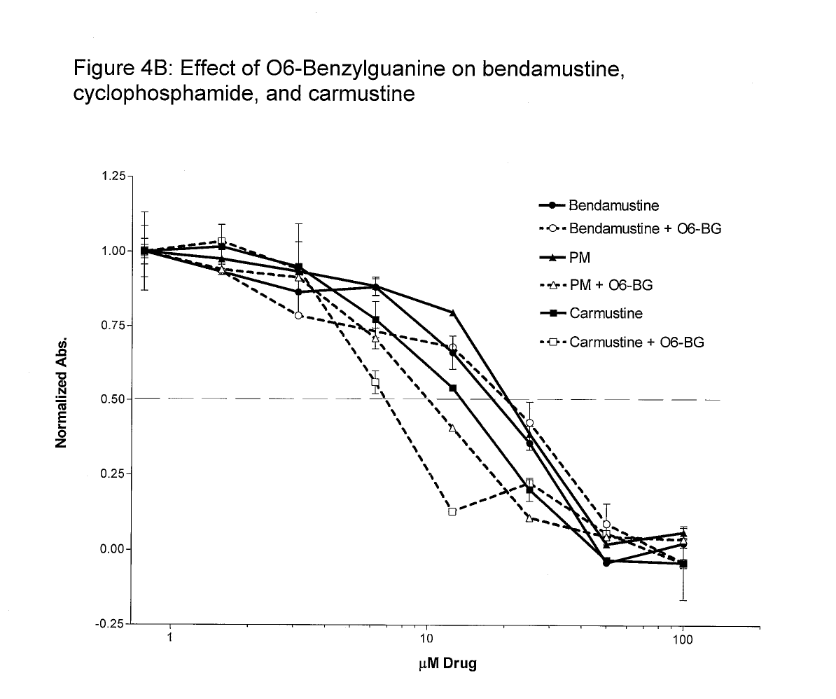

The role of the repair enzyme Ape-1, an apurininc endonuclease that plays a critical role in the base excision repair (BER) pathway in the cytotoxic activity of bendamustine and the cyclophosphamide metabolite, phosphoramide mustard, was assessed using the Ape-1 inhibitor methoxyamine. The IC₅₀ of bendamustine was reduced approximately four-fold (from approximately 50 μM to approximately 12 μM) with methoxyamine addition (FIG. 4A). In contrast, the IC₅₀ of phosphoramide mustard only changed slightly when methoxyamine was added. The results suggest that BER may play an important role in the repair of bendamustine-induced DNA damage, but not in the repair of the damage induced by cyclophosphamide.

The effect of O⁶-benzylguanine, a known inhibitor of O⁶-alkylguanine-DNA alkyltransferase (AGT) on the anti-tumor activity of bendamustine, was also tested in the SU-DHL-1 cells. The results demonstrated that the cytotoxic potency of bendamustine was not enhanced by adding O⁶-benzylguanine. Opposite results were obtained with cyclophosphamide, suggesting that unlike cyclophosphamide, bendamustine does not rely appreciably on the O₆-methylguanine-DNA methyltransferase DNA repair mechanism (FIG. 4B).

e. Bendamustine HCl Rapidly Induces the Formation of Double-Strand Breaks Resulting in Unique Cell Cycle Alterations.

To investigate the capacity of bendamustine HCl to induce double-strand breaks (DSBs), two biochemical markers were analyzed: nuclear localization of gamma-H2AX histone by immunofluorescence; and phosphorylation of H2AX at residue Ser139 by immunoblot analysis. Results confirmed that bendamustine HCl potently and rapidly induced DSBs in a variety of tumor cells, including multidrug-resistant and p53 deficient lines. Incubation with 50 μM bendamustine HCl leads to the formation of intranclear foci detectable after as few as 30 minutes. Time-course analysis showed that Ser139 phosphorylation of gamma-H2AX was detectable after 24 hours of continuous exposure to bendamustine HCL as well as after a very short exposure to the drug (30 minutes), followed by drug removal (washout). Bendamustine HCl induced phosphorylation of H2AX occurred earlier than with other 2-chloroethylamino DNA alkylators such as cyclophosphamide. Cell-cycle analysis of SU-DHL-1 lymphoma cells exposed for eight hours to 50 μM bendamustine HCl showed an average S-phase distribution increase of over 40% without an attendant G2M arrest. Exposure to equitoxic concentrations of chlorambucil and cyclophosamide increased S-phase distribution by approximately 20% and 15% respectively. These findings illustrate that bendamustine HCl can induce DNA double-strand breaks, even after a transient 30 minute exposure.

f. Bendamustine Displays a Unique Profile of Activity Using the NCI COMPARE Analysis.

Bendamustine cytotoxicity was evaluated in the 60 human cell lines of the National Cancer Institute's preclinical anti-tumor drug discovery screen (NCI screen). The NCI screen is useful for comparing relative potency of potential anti-neoplastic agents with known therapeutic agents from an extensive database of more than 45,000 compounds and natural products. The COMPARE analysis was run using the GI50 results generated with bendamustine as a “seed”. Compounds with high Pearson correlation coefficients (PCC) often have similar mechanisms of action. Bendamustine did not demonstrate a strong correlation (>0.8) in the NCI screen with any agent (Table 3, below). Out of the six top matches with bendamustine, only the methylating agent DTIC (dacarbazine) showed approximately an 80% correlative agreement (r value). In contrast, a total of 25 compounds with correlation coefficients over 0.83 were identified for melphalan, chlorambucil, or the active metabolite of cyclophosphamide. In addition, direct comparison of melphalan, chlorambucil, and cyclophosphamide sensitivity patterns in this screen demonstrated high correlation coefficients between the three drugs (0.762-0.934, data not shown). These data show a statistical agreement in sensitivity profile of the agents and a high likelihood of a common mechanism of action. The lack of correlation between bendamustine and other members of the nitrogen mustard class is compelling and reveals that bendamustine has a distinct pattern of anti-tumor activity.

TABLE 3
Closest compounds to bendamustine by NCI COMPARE Analysis

| | | Correlation (PCC)¹ |
|---|---|---|
| Compound | Mechanism of Action | GI50, TGI, or LC50 |
| DTIC, Dacarbazine | DNA Alkylator, | 0.792 (LC50) |
| | Methylating agent | |
| TOPO1B | Topoisomerase I inhibitor | 0.619 (TGI) |
| Daunomycin analog | Anthracycline, DNA | 0.574 (TGI) |
| | intercalator | |
| Melphalan | DNA Alkylator, Nitrogen | 0.550 (GI50) |
| | mustard | |
| YOSHI 864 | DNA Alkylator | 0.542 (GI50) |
| Ara-AC (Fazarabine) | Antimetabolite, DNA | 0.524 (TGI) |
| | methylation inhibitor | |

¹0 compounds show a PCC > 0.800

D. Discussion.

The results of these experiments, obtained using a variety of biological and analytical tools, demonstrate that bendamustine possesses a distinct mechanism of action when compared to other clinically used compounds that share the same “nitrogen mustard” active moiety, such as cyclophosphamide and chlorambucil.

One of the tools employed in this study was a pharmacogenomic approach, which allows the simultaneous analysis and monitoring of expression levels of thousands of fully characterized genes upon incubation of target cell lines with a selected drug, has been successfully used to elucidate the mechanism of action of other anticancer drugs. Its major advantage was the generation of unbiased information that led to the identification of a distinct mechanism of action for bendamustine, differentiating it from other DNA-alkylating agents.

With this approach, a strong classical p53-dependent stress-response “signature” was detected for bendamustine, and present, but at a greatly reduced intensity, in phosphoramide mustard- and chlorambucil-treated cells. Q-PCR analysis confirmed the gene-array analysis, validating the up-regulation of genes containing p53-responsive elements, such as p21 (Waf/Cip1) and NOXA. As an inhibitor for cyclin-dependent kinases, particularly those that function during the G₁ phase of the cell cycle, p21/Waf1/Cip1 is believed to mediate, at least in part, p53-induced G₁ arrest. The mechanisms leading to p53-induced cell cycle arrest and apoptosis have been extensively investigated and reported. Noxa encodes a Bcl-2 homology 3 (BH3)-only member of the Bcl-2 family of proteins. NOXA was shown to be a target of p53-mediated transactivation and to function as a mediator of p53-dependent apoptosis through mitochondrial dysfunction. Mouse embryonic fibroblasts deficient in Noxa showed notable resistance to oncogene-dependent apoptosis in response to DNA damage.

Activation of the p53 pro-apoptotic pathway was then confirmed by immunoblot analysis, with the detection of phosphorylated p53 (Ser15), as well as with the up-regulation of Bax. Although other nitrogen mustards have been previously reported to induce a p53-mediated stress response, bendamustine provides a stronger and more rapidly induced signal when compared to equitoxic doses of the cyclophosphamide metabolite (PM) or chlorambucil. Bendamustine was also found to induce a rapid and extensive cleavage of PARP, an enzyme that catalyzes poly(ADP-ribosylation) of a variety of proteins. Although bendamustine induces PARP cleavage, the difference between the ability of the three drugs to cause PARP cleavage in SU-DHL-1 cells was striking. This rapid induction of PARP cleavage may play a critical role in the mechanism of action of bendamustine, given the importance of PARP for DNA repair mechanisms. Indeed, in response to DNA damage, cells initially activate PARP, resulting in an increase of the accessibility of DNA to DNA repair enzymes and transcription factors. In addition, PARP has been implicated in initiating cell death by either apoptosis or necrosis.

Another major difference that emerged from the pharmacogenomic profiling of bendamustine and the other tested nitrogen mustards was the effect on expression levels of polo-like kinase 1 (PLK-1), Aurora kinases (A and B), and Cyclin B1. The mitotic checkpoint kinases PLK-1 and Aurora are involved in many aspects of cell cycle regulation, such as activation and inactivation of CDK/cyclin complexes, centrosome assembly and maturation, and activation of the anaphase-promoting complex (APC) during the metaphase-anaphase transition, and cytokinesis. Interestingly, when these checkpoint regulators are inhibited using siRNA or using targeted small molecules, potentiation of the effect of DNA-damaging drugs is observed, together with the appearance of mitotic catastrophe. Mitotic catastrophe is a form of cell death that occurs during metaphase and is morphologically distinct from apoptosis. Mitotic catastrophe can occur in absence of functional p53 or in cells where conventional caspase-dependent apoptosis is suppressed. For this reason, initiation of mitotic catastrophe is an appealing mechanism of tumor cell death, since it may also function in tumor cells that have been selected by several rounds of chemotherapy using conventional chemotherapeutic drugs. The extensive and durable DNA-damage elicited by bendamustine and concomitant inhibition of M-phase-specific checkpoints by bendamustine may trigger mitotic catastrophe in the treated cells. This may explain the clinically documented activity of bendamustine in patients refractory to cyclophosphamide and chlorambucil-containing regimens.

Efficient DNA-repair mechanisms have been demonstrated to play a critical role in the mechanism of action of DNA-alkylating drugs. Activation of discrete DNA-repair mechanisms may also confer a distinct profile of activity to drugs that share similar chemical features. The pharmacogenomic analysis described herein identified DNA-repair genes differentially regulated by bendamustine compared to phosphoramide mustard and chlorambucil. One such gene, exonuclease 1 (Exo1), is a 5′-3′ exonuclease that interacts with MutS and MutL homologs and has been implicated in the excision step of DNA mismatch repair and in the processing and repair of double-strand breaks. Exo1 has been involved in somatic hypermutation and class-switch recombination and is therefore very important in B cell function and the generation of antibodies.

To investigate further the differences in the repair mechanisms between bendamustine, cyclophosphamide, and chlorambucil, functional assays were performed. Two major mechanisms were investigated: the DNA repair protein, O⁶-alkylguanine-DNA alkyltransferase (AGT); and the apurinic/apyrimidinc endonuclease Ape-1. AGT, a ubiquitous enzyme, removes the O⁶-alkylguanine DNA adduct caused by several alkylating agents, including nitrosureas and triazenes. Clinical evidence suggests that brain tumors that express high levels of AGT, and may thus be more resistant to some DNA-alkylators such as temozolomide. The nucleoside O⁶-benzylguanine (O⁶-BG) provides a means to effectively inactivate the AGT protein. In some cell lines, benzylguanine clearly enhanced the toxicity of the activated from of cyclophosphamide. As shown here, the cytotoxic potency of cyclophosphamide, but not bendamustine, was enhanced by adding O⁶-benzylguanine, indicating that bendamustine does not induce O⁶-alkylguanine DNA adducts which can be repaired by AGT.

Ape-1/Ref-1 is an apurinic/apyrimidinic endonuclease that plays a critical role in the base excision repair (BER) pathway. BER is activated by damage induced by a variety of DNA-damaging drugs, including DNA alkylators and DNA-methylating agents, such as temozolomide. The role of Ape-1 was tested using the compound methoxyamine (MX), a specific inhibitor of its enzymatic activity. The cytotoxic activity of bendamustine was enhanced by the inhibition of Ape-1 by MX, indicating a role for BER. No changes were observed using the cyclophosphamide metabolite, underlying a major difference between the DNA-repair mechanisms activated by these drugs.

The NCI Human Tumor 60 Cell line In Vitro Screen is useful in comparing relative potency of potential anti-neoplastic agents with other known therapeutic agents. It has also been demonstrated in many cases that when pairs of compounds are found to have a high correlation coefficient between their screening results using the panel, as evaluated by the COMPARE statistical analysis program, the agents often have similar mechanisms of action. The high correlation observed for the nitrogen mustards melphalan, chlorambucil, and cyclophosphamide are all with known alkylating agents, confirming the ability of the COMPARE analysis to find common mechanisms of action. Out of the six top matches with bendamustine, only the methylating agent DTIC (dacarbazine) showed approximately an 80% correlative agreement (r value). These results reveal that bendamustine displays a distinct mechanism of action in relationship to other known alkylating agents.

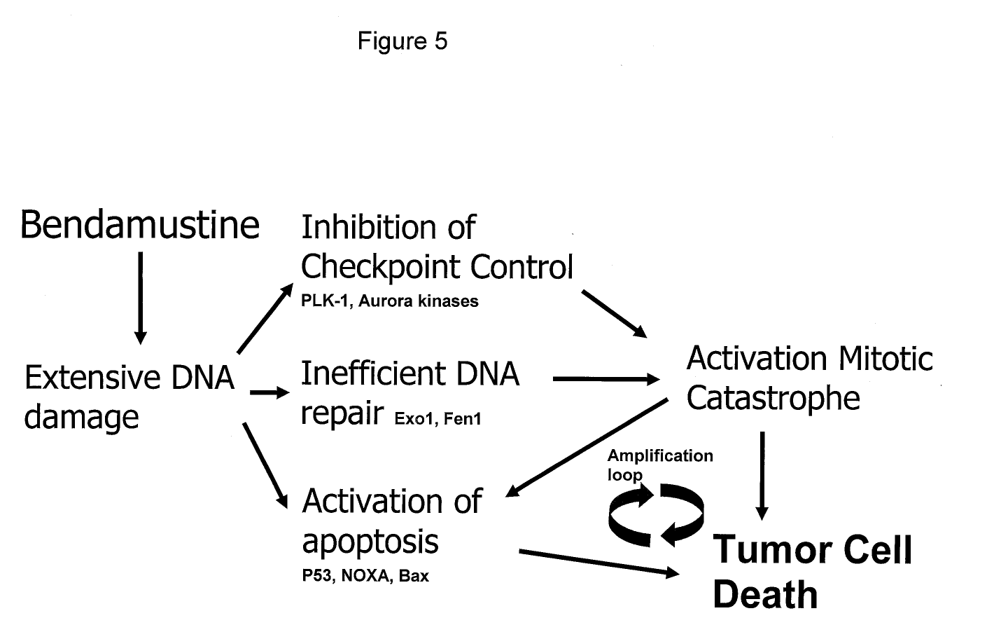

Based on the results presented in this example, the deduced mechanism of action of bendamustine is illustrated in FIG. 5. Bendamustine can efficiently enter tumor cells and induce prolonged and extensive DNA alkylation and fragmentation, probably due to the high chemical stability of the aziridinium transition state ring conferred by bendamustine's benzimidazole ring system. Bendamustine treatment results in the initiation of three main signaling pathways: 1) activation of the “canonical” p53-dependent stress pathway, resulting in strong activation of intrinsic apoptosis, which is mediated by pro-apoptotic BCL-2 family members such as NOXA and Bax; 2) activation of DNA repair mechanisms, such as the base-excision repair machinery, that are not activated by other nitrogen mustards frequently used in NHL or CLL patients; and 3) inhibition of several mitotic checkpoints, such as the kinases PLK-1 and Aurora A and B. The concomitant induction of DNA damage and inhibition of mitotic checkpoints may not allow the tumor cells exposed to bendamustine to efficiently repair the DNA damage before undergoing mitosis. Cells entering mitosis with extensively damaged DNA, or cells that cannot proceed to the “conventional” p53-dependent apoptosis, will undergo death by mitotic catastrophe. This alternative programmed cell death pathway, together with the strong activation of traditional apoptosis, indicates why bendamustine is effective in drug-resistant cells in vitro, as well as in patients carrying chemo-refractory tumors. Consequently, bendamustine treatment will represent an important addition to the armamentarium of the clinician for the treatment of patients with indolent non-Hodgkin's lymphoma and other hematologic cancers, among others.

### Example 2

### Bendamustine Activity in NHL Cells Induces the Mitotic Catastrophe Death Pathway

As described in Example 1 above, bendamustine is an alkylating agent with a distinct mechanism of action, and is undergoing clinical trials in NHL and CLL patients refractory to traditional DNA-damaging agents. Bendamustine induces unique changes in gene expression in NHL cells and displays a lack of cross-resistance with other 2-chloroethylamine alkylating agents. Quantitative PCR analysis confirmed that the G 2/M checkpoint regulators Polo-like kinase 1 (PLK-1) and Aurora A kinase (AurkA) are down-regulated in the NHL cell line SU-DHL-1 after 8 hours of exposure to clinically relevant concentrations of the drug. No changes in these same genes were observed when cells were exposed to equi-toxic doses of chlorambucil or an active metabolite of cyclophosphamide.

The ability of bendamustine to induce cytotoxicity in cells unable to undergo classical caspase-mediated apoptosis was investigated. Multi-drug resistant MCF-7/ADR cells and p53 deficient RK0-E6 colon adenocarcinoma cells were exposed for two or three days to either 50 μM bendamustine alone or 50 μM bendamustine and 20 μM pan-caspase inhibitor zVAD-fmk. Although zVAD-fmk was able to inhibit bendamustine-induced increases in Annexin-V-positive cells, microscopic analysis of nuclear morphology using the DNA stain DAPI in cells treated with either bendamustine alone or in combination with zVAD-fmk showed increased incidence of micronucleation. Multi/micro-nucleation and abnormal chromatin condensation are both hallmarks of mitotic catastrophe and have been observed in tumor cells exposed to microtubule-binding drugs such as the vinca alkaloids and taxanes. Activation of mitotic catastrophe may amplify the cytotoxicity of bendamustine and its activity in tumor cells where classical apoptotic pathways were inhibited.

### Example 3

### Fast-Acting Bendamustine Activates Potent Apoptosis and Cell Death in Lymphoma and Leukemia Cells

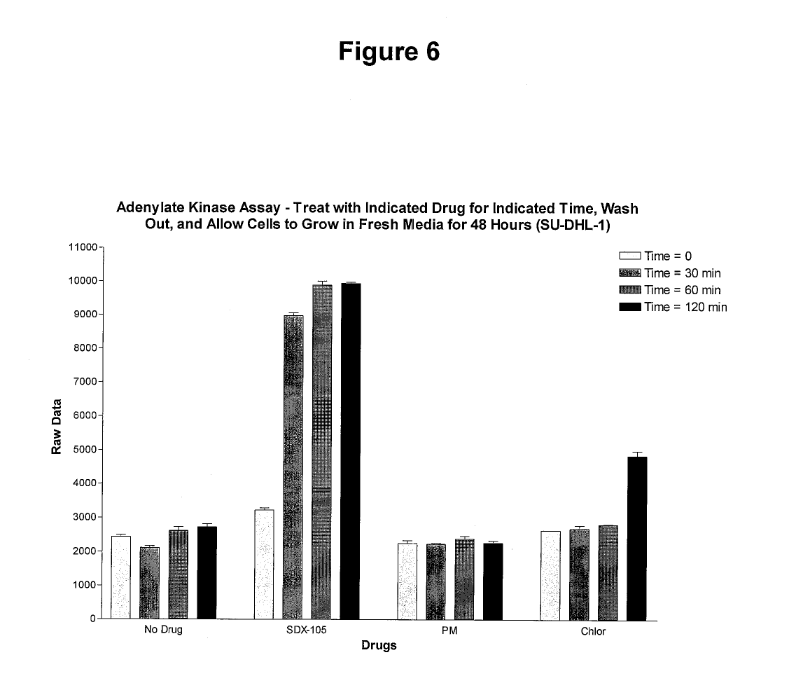

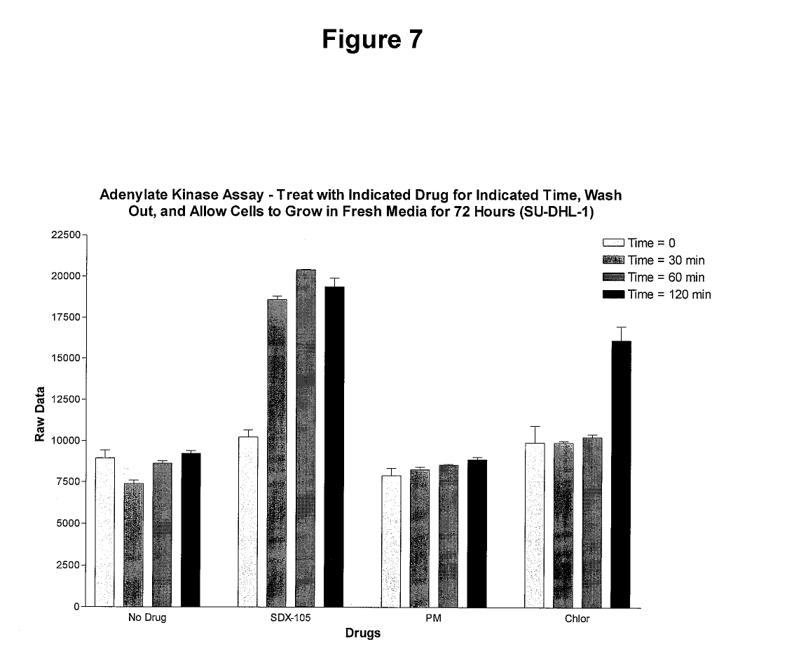

As described above, the alkylating agent bendamustine exhibits chemotherapeutic activity against drug-resistant cancers, among others, and possesses a unique mechanism of action when compared to other related anti-tumor agents. As is the case with other anti-neoplastic nitrogen mustards, bendamustine has a relatively short serum half-life in humans (approximately 2 hours), and is administered clinically by bolus intravenous infusion. The purpose of the work reported in this example was to assess the capacity of bendamustine to induce cell death and apoptosis when exposed for brief periods to cancer cells in vitro. The activity of bendamustine in such experimental models was compared to other structurally-related agents. The results obtained indicate that bendamustine exerts maximal anti-tumor activity after a brief (30 minute) exposure to cells. To obtain these results, the NHL cell line SU-DHL-1 was exposed to 50 μM bendamustine for brief periods ranging from 30 minutes to 4 hours, washed, and allowed to recover for 20 hours in drug-free media. Cells exposed to bendamustine for as few as 30 minutes displayed extensive loss of viability as measured by a variety of biological assays, including measurement of intracellular ATP and release of adenylate kinase into the supernatant at 48 and 72 hours post drug exposure (FIGS. 6 and 7). In contrast, cells treated with other members of this class of alkylating agents (here, chlorambucil, melphalan, and the cyclophosphamide metabolite phosphoramide mustard; data shown for chlorambucil and phosphoramide mustard) experienced minimal loss of viability when exposed to these agents for 30, 60, and 120 minutes. These other nitrogen mustards required a much longer exposure period (at least 4 hours) to induce a cytotoxic effect comparable to bendamustine in these assays. These findings were confirmed using an MTT-based assay in which bendamustine had a similar IC₅₀ in SU-DHL-1 and HL-60 cells at 72 hours following exposure to drug for 30 minutes, 4 hours, or 72 hours. By comparison, chlorambucil, melphalan, and phosphoramide mustard exhibited 10- to 20-fold higher IC₅₀s when incubated with these same cell lines for 30 minutes compared to continuous (72 hour) exposure.

Intracellular ATP levels were assayed using the following luciferase-based ATP assay. 10 mL of CellTiter-Glo® reagent was mixed with the appropriate amount of CellTiter-Glo substrate (per the manufacturer's instructions; Promega Corp.), and the mixture was allowed to equilibrate for ten minutes. 100 μL of this solution was then combined with 100 μL of cell-containing culture medium, and the mixture was allowed to incubate for ten minutes. Luminescence was detected using a CCD-based plate reader.

An adenylate kinase (ADK) assay was selected because as a cell membrane of a treated cell looses integrity, ADK is released into the culture medium (or, in the context of a biological sample, into the extracellular space, blood, etc. To perform the ADK assays in 96-well plates, in each test well 20 μL of supernatant from an aliquot of culture medium briefly centrifuged to pellet cells was mixed with 100 μL of the ADK reagent (20 mL Cambrex ToxiLight reagent plus the appropriate amount of Cambrex ToxiLight substrate per the manufacturer's instructions; Cambrex Corp., NJ) that had just been prepared and allowed to equilibrate for 15 min. The reaction mixture was then incubated for two minutes to allow the kinase reaction to occur. Luminescence from the samples was then read immediately in a plate reader.

Cell viability was also assessed by mixing 20 μL aliquots of the particular cell culture with 180 μL Guava ViaCount Reagent (Guava Technologies, Hayward, Calif.), diluted 1:10 dilution just prior to use. Each mixture was then incubated for five minutes. A ViaCount cell counting assay was then performed using a Guava PC Flow Cytometer, which allows the number of live cells per 1,000 total cells to be determined. Live versus dead cells were distinguished using the dye 7AAD, which can diffuse into dead or dying cells through their deteriorating cell membranes.

As described in Example 1, rapid induction of PARP (poly [ADP-ribose] polymerase) cleavage is a hallmark of bendamustine-induced cell death in NHL cells. Maximal PARP cleavage was observed in SU-DHL-1 cells exposed for as few as 30 minutes to 50 μM SDX-105 and, following drug washout, further incubated for 8 hours. No PARP cleavage was observed in cells treated in a similar manner for 30 minutes with 40 μM phosphoramide mustard, 4 μM chlorambucil, or 2 μM melphalan. The concentrations of each drug used represents equitoxic concentrations when compared to 50 μM bendamustine as measured by an MTT [3-(4,5-dimethylthiazol-2-yl)-2,5-diphenyltetrazolium bromide]-based assay after a period of 72 hours of drug exposure.

MTT assays were performed to titrate doses of the various drugs to determine the effective concentrations required to kill 50% of the treated cells. These assays were performed in 96-well plates. Concentrations ranged up to a maximum of 500 μM. In each assay, controls included untreated cells and kill control. For plates used to test cells in the “wash-out” experiments, plates were centrifuged for 5 minutes to pellet cells. Medium was then removed, the cell pellets were rinsed once with 1×PBS, and then resuspended in fresh medium. Cells were incubated with the particular dosage of drug for 3 days at 37° C. in an atmosphere containing 5.0% CO₂. After three days, 10 μL of MTT (12 mM) Reagent (5 mg/mL MTT (Promega) dissolved in fresh culture medium, filter-sterilized, stored at 2-8° C.) was added to each well. Following a four-hour incubation, 100 μL of lysis buffer (20% SDS, 0.015M HCl) was added to each well. The mixtures were placed overnight at 37° C. in an atmosphere containing 5.0% CO₂ to allow cells to lyse. The next morning, the degree of cell lysis was determined using a multiwell scanning spectrophotometer reading at 595 nm.

Comparable results were obtained by treating the human cancer cell line HL-60 with 100 μM bendamustine or 12 μM chlorambucil. Periods of exposure to the drug were 30 minutes, 1 hour, or 2.5 hours, wherein the culture medium containing drug was removed after the noted time period and replaced with fresh medium containing no drug.

Taken together, these results illustrate the unique capacity of bendamustine to activate an irreversible cell death pathway following even brief incubation with cancer cells, which distinguishes it from other related alkylating agents. Such fast-acting cytotoxicity confirms bendamustine's potent clinical activity, and indicates that it will be useful for treating various cancers, including those that are refractory to conventional chemotherapy.

### Example 4

### Clinical Data

This study evaluated the efficacy and toxicity of bendamustine in patients with NHL who have relapsed or are refractory to previous chemotherapy regimens. Patients refractory to rituximab had disease progression within 6 months of treatment.

Methods: This Phase II multicenter trial enrolled patients with relapsed indolent or transformed rituximab-refractory B-cell NHL from 17 sites in the US and Canada. Indolent histologic phenotype was seen in 84% of patients, while 16% had transformed disease. Median age of patients was 63 years (range: 38-84) and 88% had Stage III/IV disease. Patients received bendamustine 120 mg/m² IV over 30-60 minutes, days 1 and 2, every 21 days for up to 6 cycles. Response was measured using the International Working Group criteria.

Results: The intent-to-treat (ITT) population consisted of 75 heavily pretreated patients with a median of 2 prior chemotherapies. The overall objective response rate (ORR) in the ITT population was 74%; 25% had a complete response, 49% had a partial response, 12% had stable disease, and 14% had disease progression. Of 15 patients who were refractory to prior alkylator treatment (patients who progressed after at least one prior alkylator-containing therapy), 10 (67%) experienced an objective response to bendamustine. The median duration of response was 6.6 months for all patients, 9.3 months for indolent patients, and 2.4 months for transformed patients.

Conclusions: Single-agent bendamustine produced durable objective responses with acceptable toxicity, despite unfavorable prognostic features, in heavily pretreated rituximab-refractory indolent and transformed NHL patients, including those patients who were also refractory to prior alkylator treatment.

Although the invention has been described with reference to the above examples, it will be understood that modifications and variations are encompassed within the spirit and scope of the invention. Accordingly, the invention is limited only by the appended claims.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the spirit and scope of the invention as defined by the appended claims.

All patents, patent applications, and publications mentioned in the specification are indicative of the levels of those of ordinary skill in the art to which the invention pertains. All patents, patent applications, and publications, including those to which priority or another benefit is claimed, are herein incorporated by reference in their entirety to the same extent as if each individual publication was specifically and individually indicated to be incorporated by reference.

The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed:

1. A method of treating cancer, comprising determining that a patient has a cancer characterized as resistant to one or more alkylating agents and an anti-CD20 agent, comprising administering to said patient a therapeutically effective amount of bendamustine.

2. A method according to claim 1 wherein the cancer is Non-Hodgkin's lymphoma.

3. A method according to claim 1, wherein the anti-CD20 agent is rituximab.
